(19) United States
(12) Patent Application Publication
(10) Pub. No.: US 2024/0044755 A1
(43) Pub. Date: Feb. 8, 2024

(54) Method and Kit for Extracting Cells from Soil Samples

(71) Applicant: The Board of Regents of the University of Oklahoma, Norman, OK (US)

(72) Inventors: Yang Ouyang, Norman, OK (US); Jizhong ZHOU, Norman, OK (US); Aifen ZHOU, Norman, OK (US)

(21) Appl. No.: 18/166,739

(22) Filed: Feb. 9, 2023

Related U.S. Application Data
(60) Provisional application No. 63/308,219, filed on Feb. 9, 2022.

(51) Int. Cl. G01N 1/34; G01N 1/10

(52) U.S. Cl. CPC G01N 1/34; G01N 1/10; G01N 2001/1025

(57) ABSTRACT

A method of extracting viable bacterial cells from a soil sample by mixing a dispersant-surfactant solution with a soil sample to form a soil slurry, adding the soil slurry to a centrifuge tube containing a density gradient medium, centrifuging the centrifuge tube to form a solvent layer above the density gradient medium, wherein the solvent layer comprises viable bacterial cells, extracting the solvent layer containing the viable bacterial cells, combining the extracted solvent layer with a PBS solution to form a PBS-cell mixture, filtering the PBS-cell mixture to form a cell filtrate, depositing the cell filtrate into a second centrifuge tube containing a quantity of the density gradient medium, centrifuging the second centrifuge tube to form a second solvent layer comprising the viable bacterial cells, and extracting the second solvent layer from the second centrifuge medium to form a second cell filtrate comprising the viable bacterial cells.

### REFERENCE TO RELATED APPLICATIONS

The present application claims the benefit under 35 USC § 119(e) of U.S. Provisional Patent Application Ser. No. 63/308,219, filed Feb. 9, 2022. The entire disclosure(s) of the above-referenced patent application(s) are hereby expressly incorporated by reference.

### STATEMENT OF FEDERALLY SPONSORED RESEARCH

This invention was made with government support under Grant Number W911NF1920013 awarded by the Defense Advanced Research Projects Agency. The government has certain rights in the invention.

### BACKGROUND ART

Despite the abundant and diverse microorganisms in soil samples, the majority of soil bacteria and archaea remain uncultured. This hampers our understanding of the functions of these prokaryotes from soil microbial communities. Recent development of various single-cell techniques using directly extracted microbial cells provides valuable phenotypic and genomic information of these uncultured microbes. Also, such directly extracted soil microbial cells have been used for high-throughput culturing, direct quantification of bacterial abundance, and extraction of high-molecular-weight DNA. However, the relative low yield and low viability of typical soil cell extraction procedures remain a major challenge, and hence, how well the directly extractable soil cells represent the original soil samples remains largely unknown.

Intensive efforts have been invested to improve the direct microbial cell extraction efficiency from soil samples by focusing on several aspects. The first aspect has been separation of microbial cells from soil organic matter and soil particles. Physical dispersion (e.g., blending and sonication) and chemical dispersion (e.g., ionic or non-ionic buffers) are used alone or together to detach cells from soil particle surfaces. Previous studies have shown that physical and chemical dispersions largely determine cell extraction efficiencies. But the cell extraction efficiency is highly variable depending on soil textures.

A second aspect has been purification of the dispersed microbial cells. Several density gradient media have been used to purify microbial cells from soil matrices, including NYCODENZ® (Serumwerk Bernburg AG, Bernburg, Germany), HISTODENZ™ (S i gm a-Al dri ch Co. LLC, St. Louis, MO), sucrose, and sodium bromide. NYCODENZ® density gradient centrifugation is one of the most commonly used purification methods. A third aspect relates to increasing the number of extraction/purification procedures. For example, three sequential rounds of extraction recover more cells than a single-pass extraction. Despite these various methods developed, with currently reported soil cell extraction procedures, both dead and live cells are recovered after NYCODENZ® density centrifugation. Also, cell extraction efficiencies reported in literature are largely based on the total number of cells extracted that includes both live and dead cells. Therefore, to truly assess the extraction efficiency meaningful for downstream microbial phenotypic characterization, it is important to focus on the efficiency of viable cell extraction along with examining the viable microbial community compositions of cells extracted from soils.

A fourth aspect to consider has been how soil sample storage conditions affect the efficiency of viable cell extraction from soil samples. Soil samples are often stored before conducting physiological or molecular biological experiments on them. Many studies have examined the effect of soil storage conditions on microbial communities, but the results of these studies are inconsistent. Some studies have found that temperature and duration of storage have no effect on the overall microbial community composition. However, other studies have demonstrated that storage conditions significantly change soil microbial community composition. In these studies, the microbial community diversities were measured at the DNA level; however, it is unknown whether the various soil storage conditions significantly impact the viability and recovery of soil-extractable cells and soil-viable microbial community compositions.

Lastly, to evaluate cell viability, cells are often fluorescently labeled using live/dead staining reagents and quantified using microscopy or flow cytometry. However, this technique does not provide any information on the viable microbial community composition. Viability PCR has been used in cell cultures or environmental samples. Viability dyes, such as propidium monoazide or ethidium monoazide, bind to DNA from dead or compromised cells and the dye-bound DNA is degraded when exposed to certain wavelength of light, enabling the analysis of DNA originated from viable cells only.

### BRIEF DESCRIPTION OF THE DRAWINGS

FIG. 1 is a flow chart showing one embodiment of the disclosed soil cell extraction procedure. A soil sample is physically dispersed by blending and vortexing and by chemical treatment, e.g., with sodium deoxycholate and a non-ionic surfactant such as Tween® 20, to form a slurry. The slurry is subjected to a first purification step by applying the slurry to a non-ionic tri-iodobenzoic acid-type density gradient medium such as NYCODENZ® followed by centrifugation. A cell-containing band is removed from the medium and a second purification step is performed by collecting the cell-containing band in a second density gradient medium followed by centrifugation. The cell-containing band is removed from the second medium and the extracted cells are washed using PBS buffer and filtered with sterile filter (e.g., 30 μm pore size). The washed and filtered extracted cells can then be stained, e.g., using SYBR Green I and Propidium iodide, and then quantified using flow cytometry to determine cell viability. Alternatively, DNA from dead cells can be removed, e.g., by using Propidium monoazide (PMA), the DNA extraction and high-throughput sequencing can be performed.

FIG. 2A shows the yield of the soil extractable cells in different combinations of physical and chemical dispersions as well as NYCODENZ® concentrations, with two sequential rounds of cell extraction and purification. Significance of the differences in total yield of the extractable cells among different extraction combinations was tested with ANOVA (p<0.05).

FIG. 2B shows cell viability results of the extraction method used in FIG. 2A. Asterisks indicate significant difference in cell viability between the first and second cells (*p<0.05, **p<0.01, ***p<0.001). SD: sodium deoxycholate. Viability was quantified with flow cytometry of live/dead stained cells with SYBR Green I and Propidium Iodide.

FIG. 3 shows nonmetric multidimensional scaling (NMDS) ordination (stress=0.18) of the weighted UniFrac distance for microbial communities in soil or soil extractable cells with different extraction methods. The details of the combinations are presented in FIGS. 2A-2B. Left panel: without propidium monoazide (PMA) treatment. Right panel: with PMA treatment. PERMANOVA indicates significant effect of PMA, cell fractions, extraction combination (p<0.001).

FIG. 4 shows the relative abundance of several bacteria phyla (>1%) are significantly different in viable extractable cells compared to the original soil samples (p<0.05, ANOVA). PMA was used to remove DNA from dead cells. Soil cells were extracted using the combination of Blender+Tween® 20+80% NYCODENZ®. Lowercase letters indicate significant differences among soil and cells in a specific soil sample.

FIG. 5A shows the effect of storage conditions on yield of the cells extracted from four diverse soil samples. Two sequential rounds of cell extraction using the combination of Blender+Tween® 20+80% NYCODENZ® were performed. SYBR Green I and Propidium Iodide staining was used to distinguish live and dead cells. Significance of the differences was tested with ANOVA (p<0.05). Error bars represent standard deviation (n=3). Lowercase and uppercase letters (B) indicate significant differences among storage conditions for 1st CELL and 2nd CELL, respectively.

FIG. 5B shows the effect of storage conditions on viability of the cells extracted from four diverse soil samples. Two sequential rounds of cell extraction using the combination of Blender+Tween® 20+80% NYCODENZ® were performed. SYBR Green I and Propidium Iodide staining was used to distinguish live and dead cells. Significance of the differences was tested with ANOVA (p<0.05). Error bars represent standard deviation (n=3). Lowercase and uppercase letters (B) indicate significant differences among storage conditions for 1st CELL and 2nd CELL, respectively.

FIG. 6A shows Nonmetric multidimensional scaling (NMDS) ordination (stress=0.19) of the weighted UniFrac distance of microbial communities in soil bacteria and soil extractable bacteria with or without PMA treatment in fresh soil samples. PERMANOVA indicates significant effect of PMA, cell fraction, and storage (p<0.001).

FIG. 6B shows Nonmetric multidimensional scaling (NMDS) ordination (stress=0.19) of the weighted UniFrac distance of microbial communities in soil bacteria and soil extractable bacteria with or without PMA treatment in stored soil samples. PERMANOVA indicates significant effect of PMA, cell fraction, and storage (p<0.001).

FIG. 7 shows how the abundance of extracted viable cells varies according to different buffer ratios in different soil types (A), and how the abundance of extracted viable cells varies according to different chemical dispersion formulations in different soil types (B). Letters indicate a significant difference among treatments in a specific soil sample.

FIG. 8 shows the percent of spiked E. coli harboring GFP plasmid recovered by extraction with NYCODENZ® density gradient medium. Different letters indicate significant differences among soils.

FIG. 9 shows the abundance of extracted viable cells (A) and the relationship between extracted viable cells and soil organic carbon or soil total nitrogen (B) across nine soils with different properties. Different letters indicate significant differences among soils based on one-way ANOVA.

FIG. 10 shows how microbial communities of soil microorganisms and soil extractable cells change with or without PMA treatment. PERMANOVA indicated significant effect of soil, PMA, and cell fractions (p<0.001).

FIG. 11 shows how the yield of extracted viable cells after a second NYCODENZ® purification varies when various enzymes are added to the cells between the first NYCODENZ® purification and the second NYCODENZ® purification.

FIG. 12 shows the effect of different methods of physical dispersions and enzyme treatment affect extraction from small scale soil samples. There were four physical dispersions: sonication for 20 s, sonication for 40 s, vortex for 5 min, and vortex for 10 min. These physical dispersions were tested with or without enzyme treatment.

### DETAILED DESCRIPTION

Kits for isolating DNA from soil samples are commercially available to assess the microbial taxonomy and functional potential of the soil samples. However, there are no kits available for isolating living cells from soil samples. The DNA-based technologies provide limited information on phenotypic information microbes atthe single-cell level. The methods and kits of the present disclosure enable the direct extraction of viable microbial cells thereby providing valuable phenotypic and genomic information of these cells, as well as the possibility of culturing previously uncultured soil bacteria and archaea.

The direct extraction of viable microbes from soil samples is important for the application of many single-cell-related technologies. However, methods used heretofore still suffer from the challenges of relatively low yields and low cell viability. And, as noted, there is no soil microbial cell isolation kit in the market. The soil cell extraction methods and kits which are disclosed herein dramatically improve the soil cell extraction efficiency and have broad applications.

The cell isolation kits of the present disclosure have been designed for both large-scale and small-scale soil samples with all supplies and solutions in one box. The present disclosure is directed to improving the direct microbial cell extraction efficiency from diverse soil samples by focusing on the following aspects: i) dispersion of microbial cells from soil organic matters and soil particles, (ii) purification of the dispersed microbial cells, (iii) increasing the number of extraction/purification procedures, (iv) soil to buffer ratio. In the present disclosure, the microbial cell extraction procedure was applied in nine different types of soil samples, including sandy loam, loam, silt loam, and clay. In certain embodiments, the yield of soil viable cells ranged from 5×10⁷/g to 4×10⁸/g. The recovery rate tested with different soils spiked with fluoresced E. coli was 70-90%. The developed soil microbial cell isolation kit allows efficient and quick extraction of viable cells from soil samples. Taken together, the soil microbial cell extraction kits offer great potential for advancing our analyses and understanding of soil microbial ecology and the role of individual microbes.

The present disclosure is therefore directed to improve methods of extracting cells from soils, particularly regarding improved yield and viability. In one embodiment, the method uses two sequential rounds of cell extraction procedure. In experimental work, both live/dead staining and viability PCR were used to evaluate the impact of extraction procedures and soil storage conditions on the viability and microbial community composition of soil-extractable cells using soil samples having diverse ranges of physicochemical properties. By comparing different physical and chemical dispersion methods, and non-ionic tri-iodobenzoic acid-type density gradient media concentrations in soil samples having diverse physicochemical properties, a process providing high cell viability and yield was identified. In addition, using the cell extraction procedure, the effects of different soil storage conditions on yield, viability, and community composition of soil extractable cells were assessed.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility). Still further, additional aspects of the various embodiments of the instant disclosure may be found in one or more appendices attached hereto and/or filed herewith, the disclosures of which are incorporated herein by reference as if fully set out at this point.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

The term “active agent” as used herein is intended to refer to a substance which possesses a biological activity relevant to the present disclosure, and particularly refers to therapeutic and diagnostic substances which may be used in methods described in the present disclosure. “Biologically active” refers to the ability of a substance to modify the physiological system of a cell, tissue, or organism without reference to how the substance has its physiological effects.

The term “small molecule” as used herein refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

A non-limiting example of a non-ionic density gradient medium which can be used in the methods and kits of the present disclosure includes Iohexol, a non-ionic metrizoic acid-type density gradient medium having systematic name 5-(N-2,3-dihydroxypropylacetamido)-2,4,6-tri-iodo-N, N′-bis (2,3 dihydroxypropyl) isophthalamide, which is commercially available as NYCODENZ®, HISTODENZ™, and ACCUDENZ® (Accurate Chemical & Scientific Corp., Carle Place, NY).

Surfactants that may be used in the present disclosure include non-ionic surfactants and ionic surfactants (anionic, cationic, and amphoteric).

Examples of non-ionic surfactants which may be used herein include, but are not limited to, Alcohol ethoxylates, Polyethoxylated alcohols, Aliphatic alcohol ethoxylates, Alkyl phenol ethoxy a tes, Fatty acid ethoxylates, Fatty amine ethoxy a tes, Monoalkanolamide ethoxylates, Sorbitan ester ethoxylates, Ethoxylated fatty alcohols such as BRIJ™-type surfactants (Croda, Wilmington, DE), Ethylene oxide-propylene oxide block copolymers such as PLURONIC™-type and TETRONIC™-type copolymers (BASF Corp., Florham Park, NJ), and Alkyl polyglycosides, the following of which are non-limiting examples: Cetomacrogol 1000, Cetostearyl alcohol, Cetyl alcohol, Cocamide DEA, Cocamide MEA, Decyl glucoside, Decyl polyglucose, Glycerol monostearate, IGEPAL® CA-630 (Solvay, Brussels, Belgium), lsoceteth-20, Lauryl Glucoside, Maltoside, Monolaurin, Mycosubtilin, Nonidet P-40 ™(Shell Chemical Co., Houston, TX), Nonoxynol-9, Nonoxynols, NP-40, Octaethylene glycol monododecyl ether, N-Octyl beta-D-thioglucopyranoside, Octyl glucoside, Oleyl alcohol, Pentaethylene glycol monododecyl ether, Polidocanol, Poloxamer, Poloxamer 407, Polyethoxylated tallow amine, Polyglycerol polyricinoleate, Polysorbates, Sorbitan, Sorbitan monolaurate, Sorbitan monostearate, Sorbitan tristearate, Stearyl alcohol, Surfactin, Polyoxyethylene sorbitan esters, Polyoxyethylene sorbitan Octoxynol (Triton X-100™, Dow Chemical Co., Midland, MI), Polyoxyl castor oil (CREMOPHOR™, BASF), and Nonylphenol ethoxylate (TERGITOL™, Dow Chemical Co.).

Polysorbate non-ionic surfactants which can be used in the methods of the present disclosure include, but are not limited to, Polysorbate 20 (polyoxyethylene (20) sorbitan monolaurate), a polyoxyethylene sorbitol ester with lauric acid as the primary fatty acid (≥40%) with the balance comprising mainly palmitic, myristic and stearic acids, Polysorbate 40 (polyoxyethylene (20) sorbitan monopalmitate), a polyoxyethylene sorbitol ester with palmitic acid as the primary fatty acid, Polysorbate 60 (polyoxyethylene (20) sorbitan monostearate), a polyoxyethylene sorbitol ester with stearic acid as the primary fatty acid, and Polysorbate 80 (polyoxyethylene (20) sorbitan monooleate), a polyoxyethylene sorbitol ester with oleic acid as the primary fatty acid (58%). These are commercially available under the trade names TWEEN® 20, TWEEN® 40, TWEEN® 60, and TWEEN® 80, respectively (Croda).

Examples of ionic surfactants which may be used herein include, but are not limited to, anionic surfactants, cationic surfactants and amphoteric surfactants. Anionic types of surfactants include, for example, Carboxylates, Sulfonates, Petroleum sulfonates, Alkylbenzene sulfonates, Naphthalene sulfonates, Olefin Sulfonates, Sulfates, Alkyl sulfates, Sulfated natural oils and fats, Sulfated esters, Sulfated alkanolarnides, and Sulfated alkylphenols. Cationic types of surfactants include, for example, Quaternary ammonium salts, Amines with amide linkages, Polyoxyethylene alkyl amines, Polyoxyethylene alicyclic amines, N,N,N′,N′ Tetrakis substituted ethylenediamines, and Alkyl 1-hydroxyethyl 2-imidazolines. Non-limiting examples of anionic, cationic and amphoteric types of surfactants include the following: Sodium dodecyl sulfate (sodium lauryl sulfate), Sodium laureth sulfate, Lauryl dimethyl amine oxide, Cetyltrimethylammonium bromide (CTAB), Hexadecyltrimethylammonium bromide (HTAB), dodecyltrimethylammonium bromide, Polyoxyl 10, lauryl ether, Bile salts (e.g., sodium deoxycholate, sodium cholate), Methylbenzethonium chloride (HYAMINE™, Lonza Group LLC, Basel, Switzerland), N-Coco 3-aninopropionic acid/sodium salt, N-Tallow 3-Iminodipropionate, disodium salt, N-Carboxymethyl N-dimethyl N-dimethyl N-9 octadecenyl ammonium hydroxide, N-Cocoamidethyl N-hydroxyethylglycine, sodium salt, N,N-dimethyldodecylamine-N-oxides. Phosphatidylcholine, and lecithins.

Examples of compounds that can be used as dispersants in the present methods include, but are not limited to, sodium deoxycholate, sodium phosphate, sodium pyrophosphate, potassium pyrophosphate, sodium potassium pyrophosphate, tetrasodium pyrophosphate, tetrapotassium pyrophosphate, potassium citrate buffer, phosphate buffered saline (PBS), sodium bicarbonate, potassium bicarbonate, sodium potassium bicarbonate, sodium carbonate, potassium carbonate, sodium potassium carbonate, amino acids (e.g., glycine, cysteine), sodium phosphate, potassium phosphate, sodium potassium phosphate, sodium acetate, potassium acetate, sodium potassium acetate, tricine, and glycerol.

Examples of dispersant-surfactant combinations that can be used in the present methods include, but are not limited to, combinations of dispersants such as sodium deoxycholate, sodium phosphate, sodium pyrophosphate, potassium pyrophosphate, sodium potassium pyrophosphate, tetrasodium pyrophosphate, tetrapotassium pyrophosphate, potassium citrate buffer, phosphate buffered saline (PBS), sodium bicarbonate, potassium bicarbonate, sodium potassium bicarbonate, sodium carbonate, potassium carbonate, sodium potassium carbonate, amino acids (e.g., glycine, cysteine), sodium phosphate, potassium phosphate, sodium potassium phosphate, sodium acetate, potassium acetate, sodium potassium acetate, tricine, and glycerol, and surfactants such as SDS and polysorbates, such as TWEEN® 20, TWEEN® 40, TWEEN® 60, and TWEEN® 80. Such combinations include but are not limited to, sodium phosphate+SDS, sodium pyrophosphate+SDS, sodium deoxycholate+SDS, sodium phosphate+TWEEN® 80, sodium pyrophosphate+TWEEN® 80, sodium phosphate+TWEEN® 20, sodium pyrophosphate+TWEEN® 20, sodium deoxycholate+TWEEN® 20, sodium deoxycholate+TWEEN® 80, PBS+TWEEN® 20, PBS+TWEEN® 80, sodium phosphate+PBS+TWEEN® 20, and sodium phosphate+PBS+TWEEN® sodium pyrophosphate+PBS+TWEEN® 20, and sodium pyrophosphate+PBS+TWEEN® sodium deoxycholate+PBS+TWEEN® 20, and sodium deoxycholate+PBS+TWEEN® 80, sodium phosphate+PBS+SDS, sodium pyrophosphate+PBS+SDS, and sodium deoxycholate+PBS+SDS.

Where used herein, the term “large-scale soil sample” refers to a soil sample of 2.5 g or more. Where used herein, the term “small-scale soil sample” refers to a soil sample of less than 2.5 g.

### EXAMPLES

Certain embodiments of the present disclosure will now be discussed in terms of several specific, non-limiting, examples. The examples described below will serve to illustrate the general practice of the present disclosure, it being understood that the particulars shown are merely exemplary for purposes of illustrative discussion of particular embodiments of the present disclosure only and are not intended to be limiting of the claims of the present disclosure.

### Example 1

### Materials and Methods

Soils

Four surface soils (0-10 cm depth) were collected from sites in Oklahoma (Table 1). Soil A was a loam soil taken from outside of the new warming experimental plots created in 2009 (34° 58′ 45″N, 97° 31′15″ W. Soil B was a clay loam soil taken from outside of the old warming experimental plots created in 1999 (34° 58′44″ N, 97° 31′29″ W). Soil C (sandy loam soil) and Soil D (loam soil) were obtained from outside of the switchgrass plots in the Third Street site (34° 10′20″ N, 97° 04′46″ W) and the Red River site (34° 11′13″ N, 97° 05′05″ W), respectively. A soil sample (˜1 kg) was collected from each site using a hand trowel, which was sterilized by 70% alcohol before sampling. The soil sample was stored in a one-gallon zip bag, kept on ice in a cooler, and transported back to the laboratory. On arrival in the laboratory, each soil sample was homogenized, passed through sterilized 2-mm sieves, and stored at 4° C. for less than two days prior to cell extraction. Soil physical and chemical analysis was conducted in the Soil, Water and Forage Testing Laboratory at Texas A&M University, College Station, TX. To assess the effect of soil storage conditions on cell viability and microbial community composition, aliquots (˜200 g) of the soil samples were stored at 4° C., −80° C., or air-dried at room temperature for 33 to 35 days before cell extraction. To thaw soil samples stored at −80° C., soil samples were kept at −20° C. for one day, and then at 4° C. for one day.

TABLE 1
Basic physical and chemical properties of the soil samples tested

| Soil ID | Soil A | Soil B | Soil C | Soil D |
|---|---|---|---|---|
| SOC (%) | 1.23 | 1.58 | 0.8 | 0.73 |
| TN (%) | 0.11 | 0.18 | 0.08 | 0.06 |
| pH | 7.4 | 7.6 | 5.3 | 7.3 |
| Moisture (%) | 16.8 | 24.3 | 13.8 | 17.0 |
| Sand (%) | 49 | 27 | 74 | 53 |
| Silt (%) | 38 | 37 | 16 | 39 |
| Clay (%) | 12 | 35 | 9 | 7 |
| Texture | Loam | Clay Loam | Sandy Loam | Loam |

Abbreviations: SOC, soil organic C; TN, total nitrogen

Optimization of Soil Cell Extraction Procedures

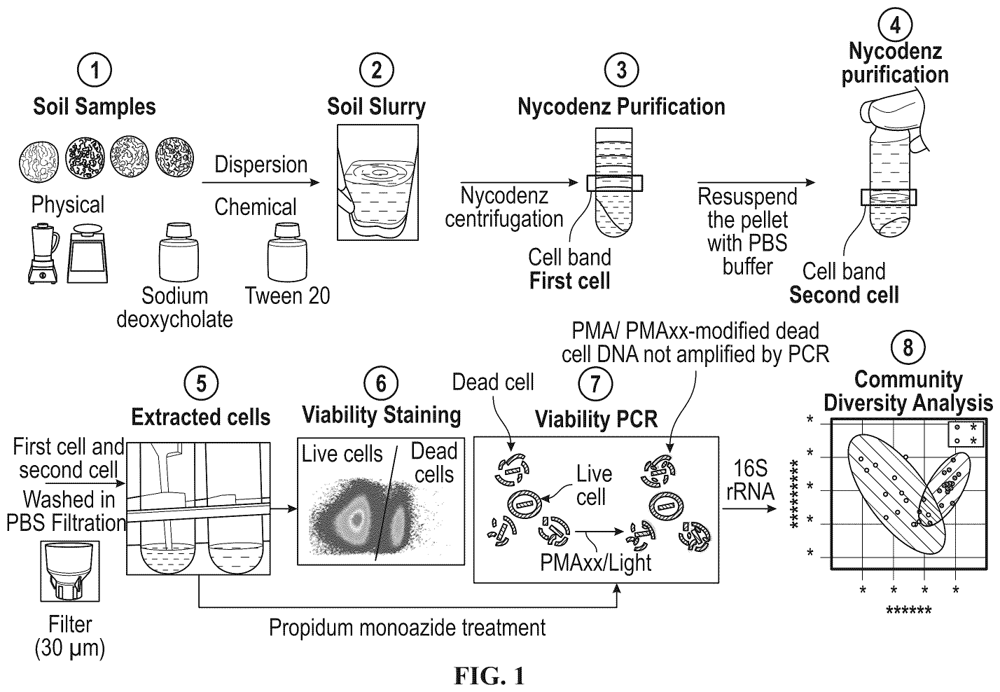

Vortex or blending were used for physical dispersion. Sodium deoxycholate (SD, 0.1% in water) or Tween® 20 (0.5% in PBS buffer) were used for chemical dispersion. Two NYCODENZ® concentrations (80% w/v and 90% w/v, in water) were tested. All buffers were sterilized by autoclaving. Two sequential rounds of extraction were conducted. Original soil samples and extracted cells were collected for DNA extraction, viability PCR, and amplicon sequencing. The overall experimental procedure is shown in FIG. 1.

For physical dispersion with blender, 40 g soil and 80 ml SD or TWEEN® 20 were added to the Waring blender (CONAIR™ 7012S, cat. 14-509-7G) and blended at highest speed (22,000 rpm) for 3 min at 1 min interval, forming a soil slurry, with 1 min incubation on ice to cool the mixture. The soil slurry was kept on ice before NYCODENZ® purification. To avoid cross contamination between soil samples, the blender was rinsed with water once followed by another rinse with alcohol (70%) after each sample. After dispersion, 20 ml of soil slurry was slowly added on the top of 18 ml 80% or 90% NYCODENZ® in a 50 ml sterile Oak Ridge centrifuge tube (three replicates) and centrifuged at 15,000×g for 40 min at 4° C. with slow acceleration and deceleration. After centrifugation, the layer containing cells above the NYCODENZ® was carefully collected using a 5 ml pipette and around 5 ml solution was transferred into a new sterile 50 ml tube. PBS buffer was added to bring the volume to 35 ml. The mixed solution was filtered with a sterile filter (MACS® SmartStrainers, 30 μm pore size, cat.130-098-458; Miltenyi Biotec, Gaithersburg, MD) into new sterile Oak Ridge centrifuge tubes to remove the large soil debris. A preliminary experiment showed that this filtration step did not lose cells. Soil-extracted cells were pelleted by centrifuging at 15,000×g for 15 min at 4° C. with slow acceleration and deceleration. The supernatant was discarded, and the cell pellet was resuspended in 5 ml of PBS buffer and designated as “1st CELL.” The soil pellet from the 1st round of NYCODENZ® purification was resuspended in 20 ml SD or Tween® 20 in each tube. The resulting slurries (60 ml) from three replicates after the first cell extraction were pooled for blending. The soil slurry was dispersed again for the second round of extraction and purification of soil cells. The cells harvested in this round was designated as “2nd CELL.”

For physical dispersion with vortex, 15 g soil and 30 ml SD were added to the 50 ml centrifuge tube. Soil slurries were then vortexed (Vortex-Genie 2, SKU: SI-0236) at the highest speed for 15 min. After vortex, 20 ml soil slurry was slowly added on the top of 18 ml 80% NYCODENZ® in a sterile Oak Ridge centrifuge tube and centrifuged at 15,000×g for 40 min at 4° C. with slow acceleration and deceleration. Soil cells were collected as described above. Two rounds of cell extraction were performed as mentioned above. There were three replicates for each soil sample.

Live-Dead Staining and Flow Cytometry

SYBR Green I and Propidium Iodide (PI) staining was used to distinguish viable and dead soil extractable cells. A SYBR Green I and PI mixture (1:3) stock solution was prepared by mixing 10 μl SYBR Green I (10,000 X stock, Invitrogen, cat. P21493) and 30 μl PI solution (20 mM, Fisher Scientific, cat. S7567) with 1 ml of sterile water in a 1.5 ml centrifuge tube. The tube was then covered with foil to avoid light and kept at −20° C. before use. Cell samples were diluted 10 or 100 times with 0.2 p.m filtered sterile PBS buffer. Then, 100 μl diluted cell samples were mixed with 10 μl SYBR Green I and PI mixture (1:3) and incubated for 20 min at room temperature in the dark.

Stained cell samples were analyzed using a Becton Dickinson ACCURI® C6 flow cytometer (Franklin Lakes, NJ, USA). Live cells stained by SYBR Green I were observed using the FL1 channel (excitation wavelength 485 nm, emission wavelength 535 nm). Dead cells stained by PI were observed using the FL3 channel (excitation wavelength 485 nm, emission wavelength 635 nm). Unstained cell samples were included as controls to exclude the signals from soil particles and debris. The threshold cutoff was set as 10,000, and each sample was run for 1 min in “slow” mode. PBS buffer was used as a blank control. Gating was used to separate positive signals from background noise. Live and dead (70% isopropanol killed) E. coli mixtures were used as control to validate the staining procedure, flow cytometer settings, and gates. The validated staining conditions or flow cytometer settings using E. coli may not be the optimal for all soil strains since soil bacteria have different cell wall structures and metabolic states. However, it is still reasonable to use the validated conditions for all cell samples in one study.

Propidium Monoazide Treatment

Propidium monoazide (PMA) was used to remove DNA from cells without intact membranes. The PMA stock solution (10 mM) was prepared by dissolving 1 mg Biotium PMA (cat. NC9734120, Fisher Scientific) in 195.7 μl Dimethyl Sulfoxide (DMSO). To prepare soil slurries for PMA treatment, 0.5 g fresh soil was added to 50 ml PBS buffer and vortexed to mix the samples. Two ml of soil slurries or cell samples in triplicates were then transferred into transparent disposable cell culture tubes (Fisher Scientific, cat. 14-956-3C). For PMA treatment group, 10 μl of PMA stock solution was added into the samples to a final PMA concentration of 50 μM. Sterile water (10 μl) was added into control group samples. All samples were incubated for 5 min at room temperature in the dark and then exposed to LED light inside the hood of Azure Biosystems C400 (RGB-cy2, 470 nm) for 20 min with manual shaking every 5 min. A preliminary experiment showed that the exposure time of 20 min was enough to remove DNA from dead cells of E. coli. One ml of PMA-treated or untreated samples were saved at −20° C. for DNA extraction.

High-Throughput Amplicon Sequencing and Raw Data Processing

DNA was extracted from the original soil samples and soil extracted cells using QIAGEN PowerSoil DNA extraction kits (Qiagen, Germantown, MD) following the manufacturer's protocol and quantified using a NanoDrop spectrophotometer (NanoDrop Technologies, Wilmington, DE).

The V4 region of 16S rRNA gene was amplified with the primer pair 515F (5′-GTGCCAGCMGCCGCGGTAA-3′) (SEQ ID NO:1) and 806R (5′-GGACTACHVGGGTWTCTAAT-3′) (SEQ ID NO:2), using a two-step PCR. The amplicons were sequenced (2×150 bp) on a Mi Seq system (Illumina, San Diego, CA, USA). Sequences were processed using a Galaxy-based sequence analysis pipeline. The forward and reverse reads were first assigned to different samples based on the barcodes. The primer sequences were then trimmed and the Brim program was used to filter the reads with a threshold quality score greater than 20 within a 5 bp window size and a minimum length of 100 bp. Forward and reverse reads with at least a 50 bp overlap and less than 5% mismatches were joined using FLASH. Sequences with ambiguous N bases were discarded. Joined sequences with lengths between 245 and 260 bp were clustered into operational taxonomic units (OTU) at the 97% identity using UPARSE. Singletons were removed. Then, taxonomic assignment was conducted through the RDP classifier with a confidence cutoff of 0.5. Sequences classified as Chloroplasts or Mitochondria were removed. An approximately-maximum-likelihood phylogenetic tree was constructed based on the representative sequences for each OTU using FastTree v.2.0.

Statistical Analyses

All statistical analyses were conducted in R software (https://www.R-project.org). Microbial 16S sequencing data were organized for diversity analysis using the R package phyloseq. After raw data processing, the retained high-quality sequences were randomly resampled to a depth of 26,095 reads per sample for 16S rRNA gene. Alpha diversity and beta diversity of the microbial communities were then calculated. Nonmetric multidimensional scaling (NMDS) and PERMANOVA were conducted to visualize and assess the Weighted UniFrac distance matrices, which incorporates both the relative abundance and phylogenetic information of each taxon, using the R package vegan. To evaluate the effect of soil storage conditions on the relative abundances of OTUs, the fold changes of all OTUs in each soil storage condition versus the fresh soil was calculated using the R package DESeq2. The p values were adjusted using the Benj amini-Hochberg correlation method. The analysis of variance (ANOVA) and Tukey's honestly significant difference (HSD) were used to characterize the statistical significance of the differences between the soil cell extraction procedures, soil storage conditions, and with or without PMA treatment.

Results

Selection of Protocol For Microbial Cell Extraction From Soil

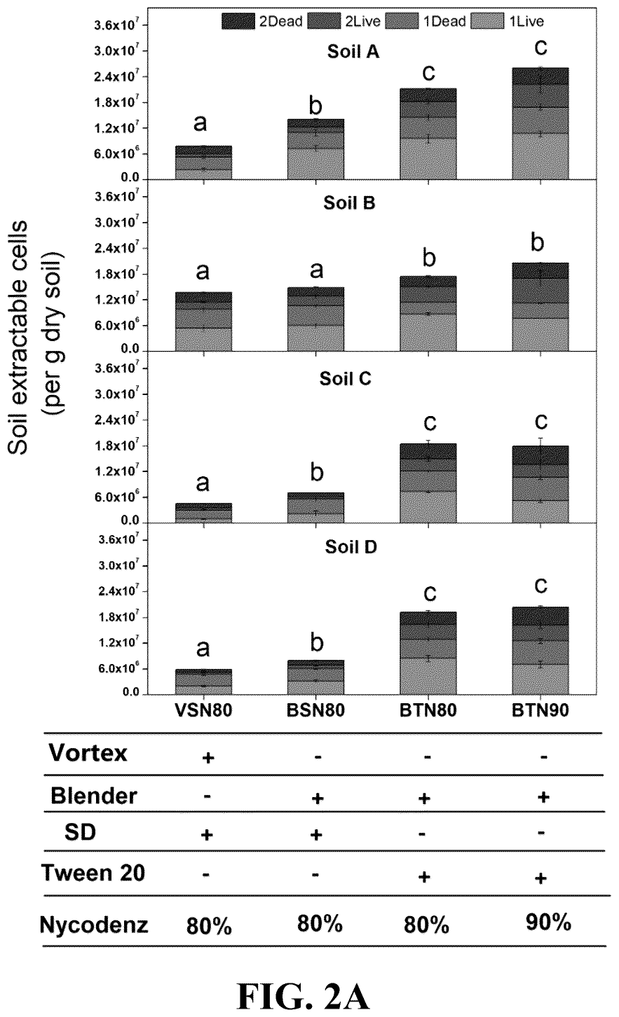

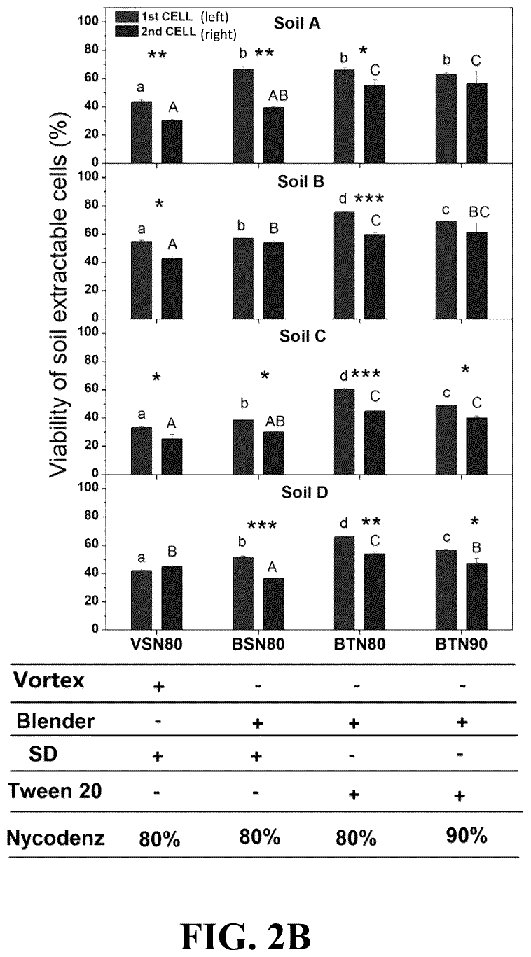

The yield and viability of soil extractable cells were evaluated using four extraction combinations of physical dispersion, chemical dispersion, and NYCODENZ® concentration. All extractions were performed with two sequential rounds (FIG. 1) in different types of soils, including loam, sandy loam and clay loam (Table 1). The total yields of soil extractable cells with two rounds of extraction and purification ranged from 4.5×10⁶ to 2.6×10⁷/g dry soil (FIG. 2A). Overall, more extractable cells were recovered in the first-round extraction (55-80% of total cells) than the second-round extraction (20-45% of total cells) in four tested soils. Cell viability was calculated as the percentage of the number of live cells in total number of cells. Higher cell viability was obtained in the 1st CELL compared with the 2nd CELL (FIG. 2B). The cell viability ranged from 42% to 75% in 1st CELL, and 25% to 61% in 2nd CELL in four tested soils (FIG. 2B). In terms of physical dispersion, blending recovered more total and viable extractable cells than vortexing. Chemical dispersion with Tween® 20 (0.5% in PBS buffer) recovered a significantly higher yield of soil extractable cells than sodium deoxycholate (SD, 0.1%). The increase of NYCODENZ® concentration from 80% to 90% had little effect on the yield and viability of soil extractable cells (FIG. 2). The combination of VSN80 (vortex+SD+80% NYCODENZ®) had the lowest viability, while the combination of BTN80 (blending+Tween® 20+80% NYCODENZ®) had the highest viability in both 1st and 2nd CELL (FIG. 2B). Considering both the cell viability and yield, the results suggested that BTN80 provided the best combination for bacterial cell extraction from soil among the conditions tested.

Microbial Community Compositions of Soil Extractable Cells Are Significantly Different From That of the Original Soil

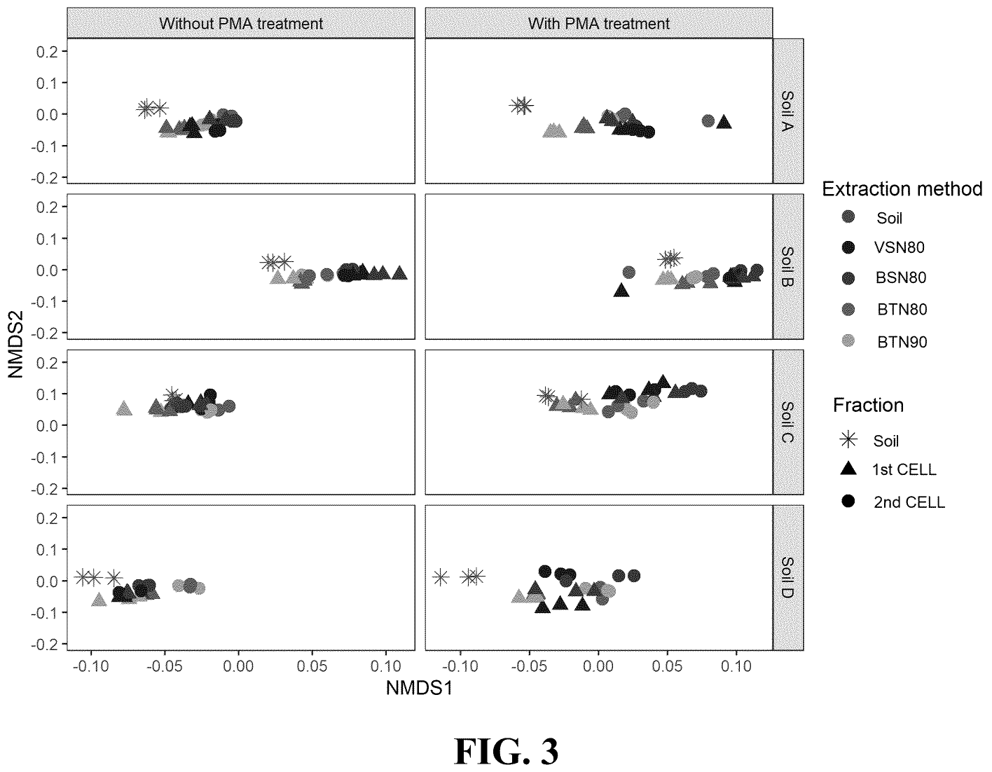

Microbial community compositions in the original soil samples and the soil-extracted cells were analyzed using 16S rRNA amplicon sequencing to assess whether the soil-extracted cells represent the diversity of the original soil microbial communities. Microbial community compositions in the extracted cells (1st CELL and 2nd CELL) were significantly different from that of the original soil samples (FIG. 3, p<0.001), regardless of the combination of cell extraction procedures used. Overall, microbial community compositions of cells dispersed by blender and TWEEN® 20 were clustered more closely with the community in the original soil samples, especially in the case of Soil C (sandy loam). Furthermore, the 1st CELL and 2nd CELL had different microbial community compositions (p<0.001). Overall, there was no difference in microbial richness (observed OTUs) among soil samples regardless of 1st CELL or 2nd CELL. The VSN80 method had the lowest richness in the 2nd CELL sample.

As expected, PMA treatment (removal of DNA from cells without intact membranes) resulted in significantly different microbial communities from both original soil samples (p=0.002) and extracted cells (p<0.001), suggesting the presence of cells without intact membranes in both soil and extracted cells (FIG. 3). In the original soil samples, PMA treatment had no significant effect on the richness and the relative abundance of abundant taxa (>1%). In contrast, in the extracted cells, PMA treatment significantly reduced the richness of both 1st and 2nd CELL, regardless of the combination of cell extraction methods used. In addition, PMA treatment exerted a stronger effect on the richness of the 2nd CELL than the 1st CELL. This was consistent with the observation of lower cell viability in the 2nd CELL, indicating a higher portion of cells without intact membranes bacteria in the 2nd CELL.

Over-Represented Or Under-represented Taxa In Soil Extractable Cells

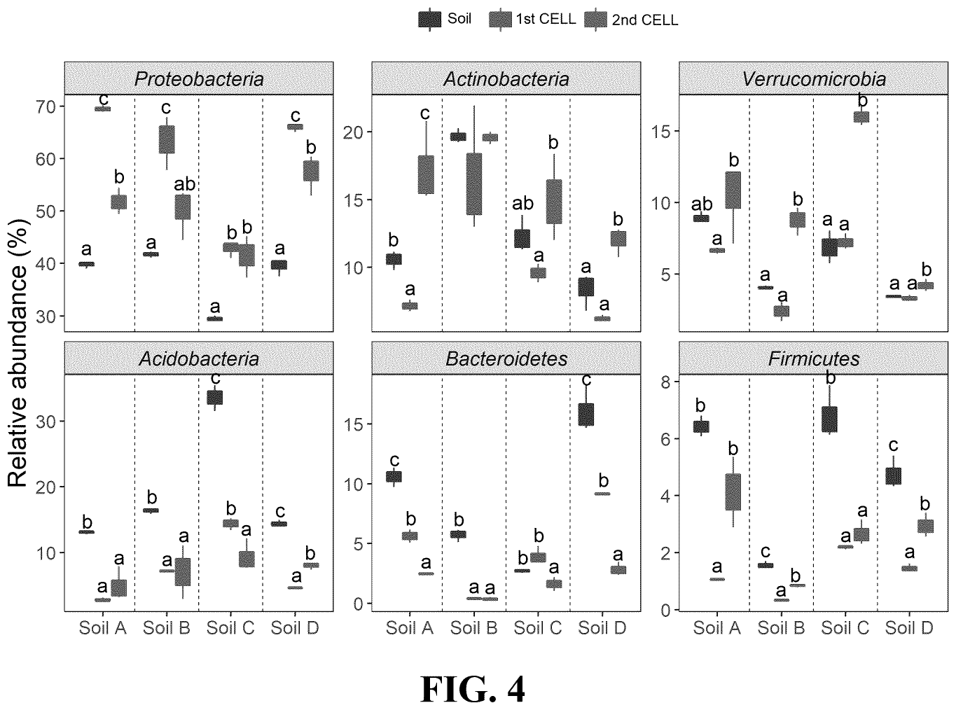

Significant differences (p=0.002, PERMANOVA) between viable (PMA-treated) and whole microbial community compositions were observed in original soil samples and soil extractable cells under all conditions tested. At the phylum level, regardless of PMA treatment, the relative abundances of key bacterial phyla in soil extractable cells were significantly different from that of the original soil samples. Also, at the phylum level, the viable communities often showed increased Actinobacteria but decreased Acidobacteria abundances in soil extractable cells. The viable microbial community was further analyzed at the phylum and genus level to uncover the source of the difference between the extractable cells and total cells in soils. Comparing the microbial communities of soils and the cells extracted with the optimal combination of BTN80 method, at the phylum level, all abundant phyla (relative abundance >1%, 13 phyla in total) in soils were recovered in extracted cells. Three abundant phyla, including Chlamydiae (in all four soils), Armatimonadetes (in Soil D), and Parcubacteria (in Soil C), were observed in the extracted cell populations only, suggesting an increased detectability of certain phyla in extractable cells. In addition, six abundant phyla were found in both the total and extractable cell populations but with significantly different abundances (FIG. 4). Proteobacteria were over-represented in both 1st CELL and 2nd CELL, with the highest relative abundance in 1st CELL. Abundances of Actinobacteria and Verrucomicrobia were generally lower in 1st CELL and higher in 2nd CELL compared with soil. Three phyla including Acidobacteria, Bacteroidetes, and Firmicutes were under-represented in the extracted cell population. Here, lower relative abundances were obtained in both 1st CELL and 2nd CELL compared with the total soil population for all three phyla, but Firmicutes had a higher abundance in 2nd CELL than 1st CELL. At the genus level, many genera were missing in the extracted cell population. The abundant genera with a relative abundance >0.1% across four soils was focused on. Among the abundant genera, 13 genera were considered to be “hard-to-extract” taxa, since they were present in soil samples but absent in cells extracted from at least three soil samples. Most of these microbes belong to Bacteroidetes, Firmicutes, and Proteobacteria (Table 2).

TABLE 2
The relative abundances (%) of hard-to-extract Genera in soil samples

| Genus | Phylum | SoilA | SoilB | SoilC | SoilD | Potential traits |
|---|---|---|---|---|---|---|
| Virgisporangium | Actinobacteria | 0.14 | 0.31 | ND | 0.41 | Sporulation |
| Ferruginibacter | Bacteroidetes | 0.66 | 0.13 | 0.12 | 1.10 | Attachment to SOM |
| Flavisolibacter | Bacteroidetes | 0.30 | 0.31 | 0.49* | 1.12 | Attachment to SOM |
| Flavitalea | Bacteroidetes | 0.14 | 0.21 | ND | 0.53 | Attachment to SOM |
| Niastella | Bacteroidetes | 0.40 | 0.21 | 0.18* | 0.61 | Filamentous shape |
| Solitalea | Bacteroidetes | 0.24 | 0.19 | 0.12 | 0.51 | Filamentous shape |
| Sporosarcina | Firmicutes | 1.06 | 0.13 | 0.94 | 1.46 | Sporulation |
| Tumebacillus | Firmicutes | 0.76 | ND | 0.27 | 1.19 | Sporulation |
| Haliangium | Proteobacteria | 0.44 | 0.61 | 0.24 | 0.50 | Cell aggregation |
| Labrys | Proteobacteria | 0.13 | 0.13 | ND | 0.17 | Cell aggregation |
| Phaselicystis | Proteobacteria | 0.51 | 0.16 | ND | 0.22 | Cell aggregation |
| Piscinibacter | Proteobacteria | 0.26 | 0.17 | 0.17 | 0.57* | Cell aggregation |
| Sorangium | Proteobacteria | 0.28 | 0.15 | 0.11 | 0.30 | Myxobacteria, cell aggregation |

Hard-to-extract Genera were defined as genera that present in total soil DNA extracts but absent in the DNA of extracted cells in at least three soil samples with relative abundance cutoff of 0.1%. ND indicates an absent genus in a specific soil sample.
*indicates the genus was detected in the extracted cells.
SOM = soil organic matter.

Effects of Soil Storage Conditions On Yield and Viability of Soil Extractable Cells

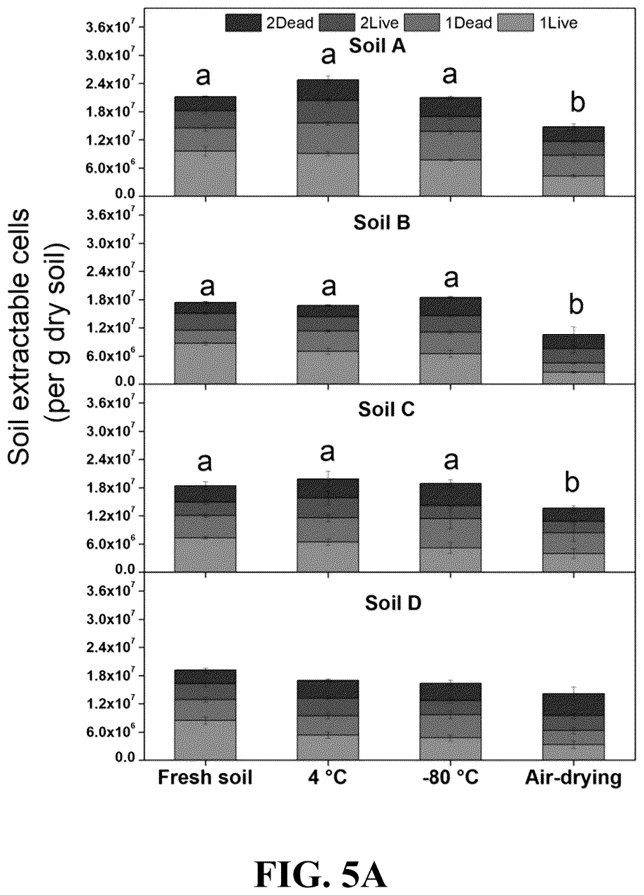

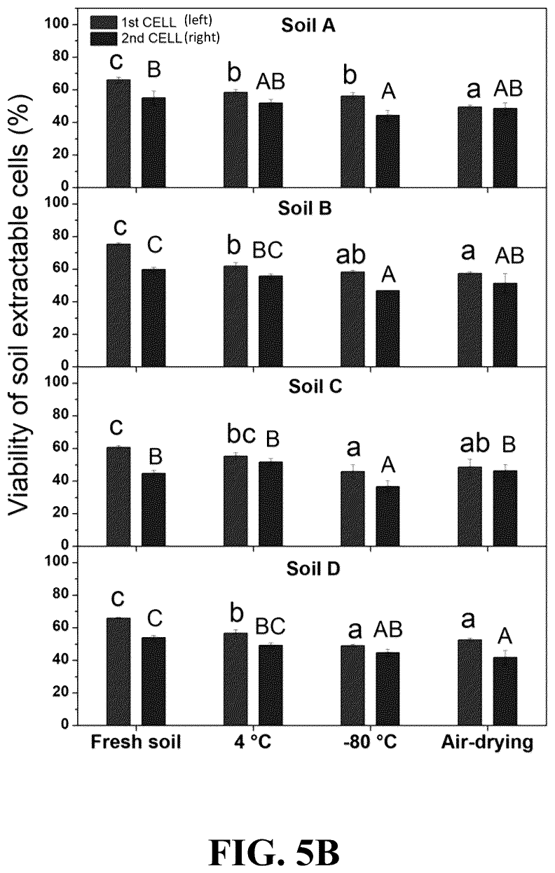

Using the selected cell extraction procedure (BTN80), the effect of soil storage conditions (4° C., −80° C., and air-drying) on the yield and viability of soil extractable cells was assessed. Storage at 4° C. or −80° C. had no effect on the yield of the total extracted cells compared with fresh soil (FIG. 5A), while air-drying at room temperature significantly reduced the yield. Viability of 1st CELL decreased under all storage conditions (FIG. 5B), but it showed the least drop under the 4° C. storage condition (8.8-18% drop compared to fresh soil). For 2nd CELL, no significant difference was found between the fresh soil and soil stored at 4° C.; however, significantly decreased cell viability was observed in soil samples stored at −80° C. or subject to air-drying. Taken together, the viability of soil extractable cells was more sensitive to soil storage conditions due to cell death. Considering both cell yield and viability, it was determined that 4° C. is the best condition to store soil samples, −80° C. is the 2nd choice, and air-drying is not recommended for viable cell extraction and isolation.

Storage Conditions Changed Microbial Community Compositions of Soil and Soil Extractable Cells

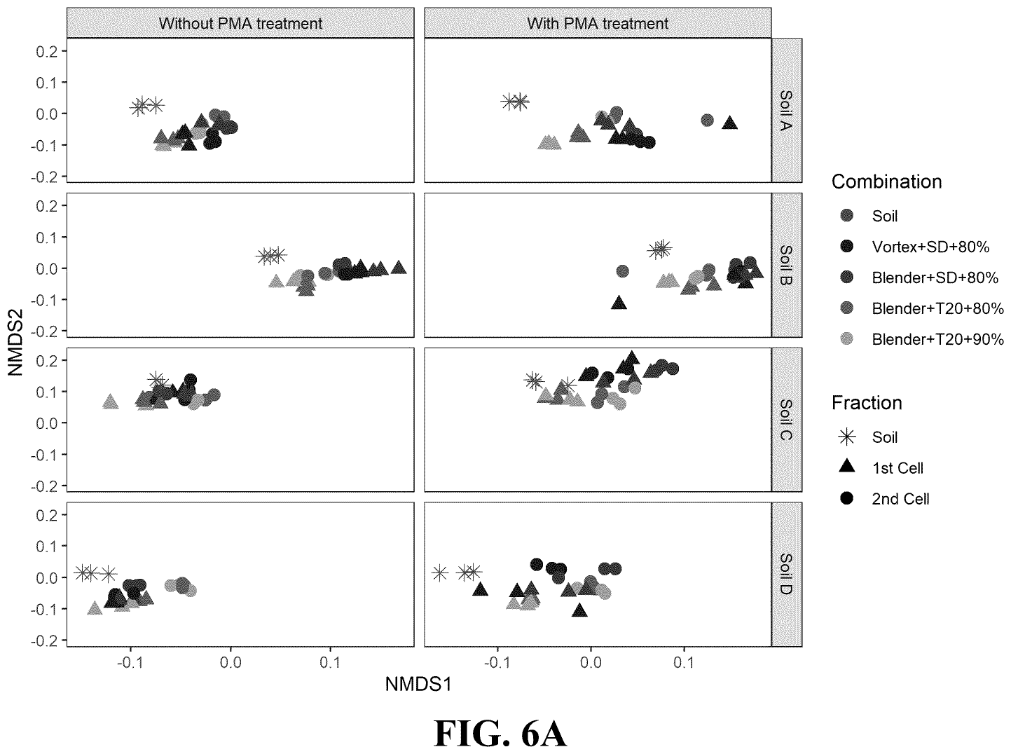

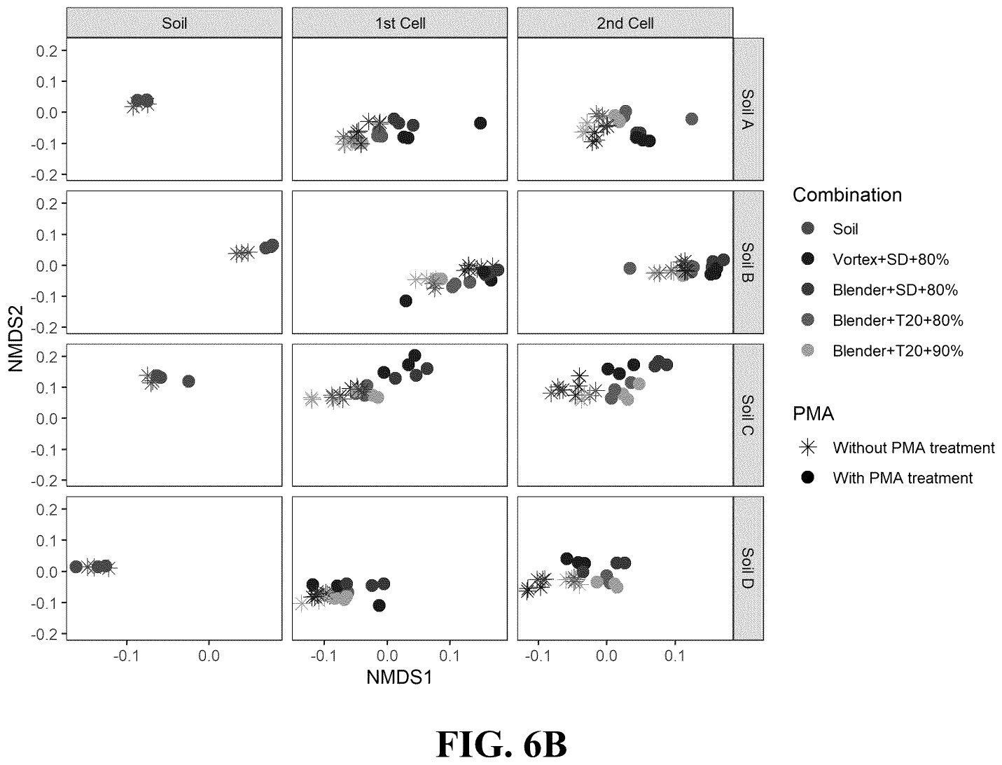

Both viable and total microbial community compositions of soil and soil extractable cells were significantly changed by the three storage conditions (FIG. 6). Air-drying had a stronger effect than storage at 4° C. or −80° C. Overall, microbial community compositions of soil samples stored at 4° C. were clustered more closely with that of fresh soil. In addition, the richness of both total and viable microbial communities in soil and extracted cells decreased under the three storage conditions compared with that of fresh soil. Air-dried samples generally had the lowest richness in soil and 1st CELL. There was no difference in the richness of the 2nd CELL among the three storage conditions. Compared with the total microbial community, the viable microbial community was more sensitive to the storage conditions (FIG. 6). Similar as the fresh soil, PMA treatment had a significant effect on the microbial community composition of the total soil population (p<0.001) and extracted cell population (p<0.001) under different storage conditions (FIG. 6). In addition, samples treated with PMA showed significantly (p<0.001) lower microbial richness in frozen and air-dried samples.

The viable microbial community compositions at phylum and OTU level were then analyzed to uncover differences derived from soil storage conditions. In terms of microbial communities of soil samples, the abundant phyla generally showed no difference between fresh soil and soil stored at 4° C. However, relative abundances of several bacterial phyla showed significant changes (p<0.05) in frozen or air-dried soil samples. For example, relative abundances of Proteobacteria, Acidobacteria, Bacteroidetes, and Verrucomicrobia were generally significantly lower, while Actinobacteria and Firmicutes were generally higher, in soil samples stored at −80° C. or air-drying than in fresh soil or soil stored at 4° C. (data not shown). Similarly, at OTU level, less responsive OTUs were observed in soil samples stored at 4° C. than soils stored at −80° C. or air-drying (data not shown). Most of the responsive OTUs, defined as significant changes of relative OTU abundances and calculated as the loge-fold change, under storage at −80° C. or air-drying were in the phyla of Proteobacteria, Acidobacteria, Actinobacteria, Bacteroidete, and Verrucomicrobia. Significant changes of viable microbial community compositions at both phylum and OTU level by soil storage conditions were also found in soil extractable cells. In the 1st CELL, most of the abundant phyla (>1%) showed no difference between fresh soil and soil stored at 4° C., except for the decreased relative abundance of Acidobacteria and increased relative abundance of Actinobacteria in Soils C and D. Interestingly, more than 700 responsive OTUs in Proteobacteria were detected in 1st CELL stored at 4° C. across the four soil samples, although this phylum showed no difference. Similar to the total soil microbial communities, the relative abundances of Proteobacteria and Acidobacteria significantly decreased, while Actinobacteria increased in 1st CELL extracted from soil samples stored at −80° C. or air-dried. Not surprisingly, a majority of the responsive OTUs under storage at −80° C. or air-drying belonged to these three phyla. Although there was limited difference at the phylum level, more responsive OTUs were obtained than from that of original soil samples (2,283 OTUs in 1st CELL vs. 1,874 OTUs in soil), suggesting that these extracted cells were more sensitive to soil storage, especially at −80° C. and air-drying. In the 2nd CELL, there was a large variance in the relative abundance of phyla extracted from the air-dried soil samples. Proteobacteria and Verrucomicrobia had decreased abundances, while Actinobacteria and Firmicutes had increased abundances in 2nd CELL extracted from soil samples stored at −80° C. or air-dried. Similarly, most responsive OTUs under the storage conditions were in these four phyla. Overall, less responsive OTUs were detected in the 2nd CELL (1146 OTUs) than that from original soil and 1st CELL.

Discussion

Extraction of microbial cells from soil is a critical step for the application of many microbial discovery campaigns, including those performed at single-cell resolution. For example, microbial diversity in soil has been extensively mined for the discovery of novel antibiotics, anti-cancer compounds, enzymes, and organisms. Previous work on extraction methods often focused on the improvement of cell recovery efficiency evaluated at the total number of cells instead of total number of viable cells. In the present work, it was examined how extraction procedures and soil storage conditions affected the viability and microbial community compositions of the soil extractable cells using four soil samples with different soil types (loam, sandy loam, and clay loam). The combination of soil cell extraction method, namely the use of blender+TWEEN® 20+80% NYCODENZ®, had the highest cell viability and yield among the conditions tested. Repeated cell extraction could improve the overall cell yield, but the viability could be compromised in the second round of cell extraction from soil. Storage of soil samples at −80° C. or air-drying significantly decreased the cell viability and/or yield. Our result shows that there is a significant difference between total cells extracted and total viable cells extracted under all conditions tested in the present study, and that for some microbiological applications, focusing on viable cell extraction is critical rather than total cells.

Our test of extraction procedures demonstrated the positive effect of the combination of ionic and non-ionic buffers as well as repeated extraction, but not the increase of NYCODENZ® concentration, in improving the cell yield. In terms of physical dispersion, it was found that blending had higher cell yield and viable than vortex. Although blending is a harsh dispersion process, the three one min-short blending duration and one min-incubation on ice between intervals might help to maintain the cell viability. In contrast, 15 min continuous vortex at room temperature might be very disruptive for cell integrity. Whether chemical dispersion improves cell yield is still in debate. Although some studies showed that chemical dispersion had no effect on the cell yield, other studies indicated that sodium deoxycholate (SD, a mild detergent) was the best buffer for a broad range of soil textures compared with six ionic or non-ionic buffers. The combination of ionic and non-ionic buffers (TWEEN® 20 in PBS buffer) resulted in significantly higher cell yield and viability than SD in our study, suggesting that combined chemical buffers may destroy different forms of microbial attachment to soil particles. Repeated extraction contributed to a high amount (20-40% higher) of total soil extractable cells. This finding is consistent with previous studies about high recovery of cells with repeated extraction. However, it should be noted that repeated extraction is time-consuming, and more importantly, it significantly reduces the cell viability. The reduction of the cell viability may be contributed by the damage from repeated physical dispersion, or the recovery of cells with compromised membrane integrity in the second round of cell extraction. Our results do not support the hypothesis that a further increase in NYCODENZ® concentration could improve cell yield. The density of vegetative cells often ranges from 1.11 to 1.20 g/ml. The 80% NYCODENZ® has a density of around 1.426 g/ml, which is high enough to recover most vegetative cells.

Analysis of the microbial community compositions demonstrated that the soil extractable cells with two rounds of blending and TWEEN® 20 dispersion followed by NYCODENZ® purification represented the original soil microbial communities better than other combinations tested. Different microbial communities were recovered in each cell extraction method combination and each round of extraction, suggesting that each physical and chemical dispersion as well as each round of extraction were effective for certain microbial groups. To recover microbial cells better representing the original microbial communities in soil samples, multiple combinations of physical and chemical dispersions are recommended. In one embodiment, combining the 1st CELL and 2nd CELL even better represents the original soil microbial community. The cause of the bias in microbial community of soil extracted cells compared to that in the original soil has been suggested to be the often over-represented by Proteobacteria, Acidobacteria, and Verrucomicrobia, while usually under-represented by Actinobacteria and Firmicutes in cells extracted by a single-pass extraction. In the present work, it was observed that Proteobacteria was indeed over-represented, while the other above-mentioned four phyla were actually under-represented in 1st CELL. However, in 2nd CELL, relative abundances of Actinobacteria, Verrucomicrobia, and Firmicutes were generally over-represented. These results further highlight the importance of repeated cell extraction in recovering a more similar soil microbial communities as that in the original soil.

Soil prokaryotes include several classes of hard-to-extract microbes including those that can form spores, are filamentous in cell shape, produce extracellular compounds, and biofilm formers. For instance, genera Virgisporangium, Sporosarcina, and Tumebacillus (Table 1) are spore formers. Due to the potential higher density of these spores than NYCODENZ®, some spores may not be extracted with NYCODENZ® density centrifugation. Most of these hard-to-extract genera in Bacteroidetes belong to the family Chitinophagaceae. These microbes may be strongly attached to chitin particles in order to degrade chitin or other soil organic matter. Among this group, Niastella and Solitalea have a filamentous cell shape. Their tight binding to soil organic matters and particles probably makes them harder to extract. Although the Proteobacteria was over-represented, five abundant genera were not recovered in the extracted cells. Most of these genera have a biofilm lifestyle and form cell aggregates, therefore using a density gradient medium that has higher density than NYCODENZ® may have to be used to extract these cells. In order to recover the hard-to-extract genera, certain non-limiting embodiments of the present disclosure recommend using optimized physical and chemical dispersions to recover the most easy-to-extract species in the 1st round cell extraction. Stronger detergents and digestive enzymes could then be used to disperse tightly attached cells in the 2nd round extraction, while spores could be extracted using sodium bromide (NaBr) density gradient centrifugation in the 3rd round extraction.

Extracting cells immediately from fresh soil is ideal to investigate their microbial community composition and function. However, often soil cannot be processed immediately due to many reasons, including large number of soil samples that needs to be tested, transportation requirement from field sites to laboratories, and also due to difficulties in keeping cells alive once they are extracted from soil and thus the need to extract cells at the time of need, to name a few. Analysis of the viable microbial communities from fresh and stored soil samples provided evidence of the effect of soil storage conditions on the viability of soil extractable cells. Our analyses show that both the total and viable soil microbial communities were changed due to storage conditions. Based on our analysis, short-term storage at 4° C. is recommended because only small changes in microbial community composition and yield of soil extractable cells were observed compared with fresh soil. In contrast, storage at −80° C. or after air drying had significant negative impact on the cell viability and community composition. Other studies also showed that freezing and air-drying significantly changes microbial biomass and community composition. Freezing or drought change soil water potential and water film thickness, which causes a strong physiological stress on soil microbes. Microbes may enter a dormant state or die after freezing or drought. Indeed, relative abundances of Gram-positive bacteria (Actinobacteria and Firmicutes), which are tolerant to water stress, increased in 1st CELL or 2nd CELL. In contrast, relative abundances of phyla such as Proteobacteria, Acidobacteria, Bacteroidetes, and Verrucomicrobia, and OTUs in these phyla decreased in cells extracted from −80° C. stored or air-dried soil samples. Considering the changes in microbial community and reduction in viability and richness, storage at −80° C. or after air-drying should be avoided for viable soil cell extraction.

Conclusions

Obtaining a large number of viable cells that represent the microbial community in soil as close as possible is often the first step for many microbiological applications. It was found that implementation of a protocol that features the use of a blender+Tween®20+80% NYCODENZ® provides the optimal combination for direct viable soil bacteria extraction among conditions tested in the present study. First and second rounds of cell extraction resulted in different microbial community compositions, which were also different from the original soil microbial community. Several abundant genera were identified that were hard-to-extract from soil. Sporulation, filamentous cell shape, EPS production, and biofilm lifestyle are the traits that potentially affect the extraction efficiency. Future efforts will test combinations of ionic and non-ionic detergents as well as digestive enzymes to improve the recovery rate of the extractable cells that better represent the original soil microbial communities. Using the optimized cell extraction procedure, the effect of soil storage conditions (4° C., −80° C., and air-drying) on yield and viability of soil extractable cells was also assessed. Soil storage at 4° C. and −80° C. had no effect on the cell yield, while air-drying significantly reduced the yield. The abundance and community composition of soil viable cells were very sensitive to all soil storage conditions. If fresh soils cannot be processed, short-term storage at 4° C. is recommended, while freezing at −80° C. or air-drying at room temperature should be avoided for viable soil cell extraction purposes.

### Example 2

### Materials and Methods

Soils

Nine surface soils (0-10 cm depth) were collected from sites in Oklahoma and Texas (Table 1). Soil A, Soil B, Soil C, and Soil D were described in Example 1. Soil E and Soil F were from Prairie grassland soil and agricultural soil, respectively. Soil G was a forest soil sampled in Thunderbird State Park, Oklahoma. Soil H and Soil I were from Texas. The same soil sampling method was used as in Example 1. Briefly, a soil sample (˜1 kg) was collected using a hand trowel, kept on ice in a cooler, and transported back to the laboratory. On arrival in the laboratory, each soil sample was homogenized, passed through sterilized 2-mm sieves, and stored at 4° C. for less than one month prior to cell extraction. Soil physical and chemical analysis was conducted in the Soil, Water and Forage Testing Laboratory at Texas A&M University, College Station, TX.

Selection of Soil Cell Extraction Procedures For Large-Scale Soil Samples

In Example 1, the use of a blender with PBS+TWEEN® 20+80% NYCODENZ® provided the best combination among tested conditions. Briefly, 40 g soil and 80 ml 0.5% TWEEN® in PBS were added to the Waring blender (CONAIRTM 7012S, cat. 14-509-7G) and blended at highest speed (22,000 rpm) for 3 min at 1 min interval with 1 min incubation on ice to cool the mixture. After dispersion, 20 ml soil slurry was slowly added on the top of 18 ml 80% NYCODENZ® in a 50 ml sterile Oak Ridge centrifuge tube and centrifuged at 15,000×g for 40 min at 4° C. with slow acceleration and deceleration (three replicates). The layer containing cells above the NYCODENZ® was then carefully collected using a 5 ml pipette and around 5 ml solution transferred into a new sterile 50 ml tube. PBS buffer was added to bring the volume to 35 ml. The mixed solution was filtered with a sterile filter (MACS® SmartStrainers, 30 μm pore size, cat.130-098-458) into new sterile Oak Ridge centrifuge tubes to remove the large soil debris. Soil-extracted cells were pelleted by centrifuging at 15,000×g for 15 min at 4° C. with slow acceleration and deceleration.

This example further refined and modified the soil cell extraction procedures using four soils with different textures. The textures of these four soils were sandy loam (Soil A), clay loam (Soil B), silt loam (Soil E), and clay (Soil I). Soil-to-buffer ratios, combined physical dispersions, combined chemical buffers, and enzyme pretreatment before the second round of soil cell extraction were tested. After NYCODENZ® centrifugation, all supernatant above the NYCODENZ®, rather than only the layer containing cells, was collected.

First, the soil-to-solution buffer ratios were tested with the use of a blender+TWEEN® 20+80% NYCODENZ®. Five soil-to-solution buffer ratios were tested: 1:2 (e.g., 50 g soil :100 ml 0.5% TWEEN® in PBS), 1:5, 1:10, 1:20, 1:50. Using the best soil to solution ratio, four blending times were tested. Also, the combined physical dispersions (shaking and blending) were compared with blending. For the combined physical dispersions, the soil slurry was shaken for 30 min at 4° C. with a speed of 100 rpm, followed by blending as described above. It was found that the cell yield was not improved using the combined physical dispersions. Henceforth blending was used for physical dispersion. Five different chemicals were mixed with 0.5% tween 20: 0.1% sodium deoxycholate, PBS buffer, 0.1% sodium deoxycholate+PBS buffer, 50 mM sodium pyrophosphate, 50 mM sodium pyrophosphate+PBS buffer. Lastly, the performance of enzyme pre-treatment before the second round NYCODENZ® purification was evaluated. Seven enzymes, including α-amylase (Sigma-Aldrich, cat. A6814), (β-galactosidase (Sigma-Aldrich, cat. G5160), pectinase (Sigma-Aldrich, cat. P4716), lipase (Sigma-Aldrich, cat. L3126), cellulase (Sigma-Aldrich, cat. C2605), protease (Sigma-Aldrich, cat. P5380), and DNase (Sigma-Aldrich, cat. DN25) were separately added into the soil slurry and incubated for 30 min at 37° C. before the second round NYCODENZ® purification. A cocktail of enzymes (α-Amylase, (β-Galactosidase, and Cellulase) were also added into the soil slurry and incubated for 30 minutes before the second round NYCODENZ® purification.

Selection of Soil Cell Extraction Procedures For Small-Scale Soil Samples

On the basis of soil cell extraction procedure of large-scale soil samples, the best soil cell extraction method from small-scale soil samples was also tested. 1 g soil and 20 ml sodium pyrophosphate with 0.5% tween were added to a 50 ml sterile Oak Ridge centrifuge tube. A cocktail of enzymes (α-Amylase, (β-Galactosidase, and Cellulase) was added into the soil slurry and shaken for 60 minutes at 37° C. with a speed of 100 rpm. After enzyme treatment, the soil slurries were further dispersed by sonication and vortexing. Two timings were selected: 20 s and 40 s, with 5 s intervals. For vortexing, the maximum speed for 5 min and 10 min was used.

The soil slurry was slowly added on the top of 18 ml 80% NYCODENZ® in a 50 ml sterile Oak Ridge centrifuge tube and centrifuged at 15,000×g for 40 min at 4° C. with slow acceleration and deceleration (three replicates). The cell-containing layer above the NYCODENZ® was then carefully collected using a 5 ml pipette and around 18 ml solution was transferred into a new sterile 50 ml tube. PBS buffer was added to bring the volume to 35 ml. The mixed solution was filtered with a sterile filter (MACS® SmartStrainers, 30 μm pore size, cat.130-098-458) into new sterile Oak Ridge centrifuge tubes to remove the large soil debris. Soil-extracted cells were pelleted by centrifuging at 15,000×g for 15 min at 4° C. with slow acceleration and deceleration.

Live-Dead Staining and Flow Cytometry

SYBR Green I and Propidium Iodide (PI) staining was used to distinguish viable and dead soil extractable cells. A SYBR Green I and PI mixture (1:3) stock solution was prepared by mixing 10 μl SYBR Green I (10,000×X stock, Invitrogen, cat. P21493) and 30 μl PI solution (20 mM, Fisher Scientific, cat. 57567) with 1 ml of sterile water in a 1.5 ml centrifuge tube. The tube was then covered with foil to avoid light and kept at −20° C. before use. Cell samples were diluted or 100 times with 0.2 p.m filtered sterile PBS buffer. Then, 100 μl diluted cell samples were mixed with 10 μSYBR Green I and PI mixture (1:3) and incubated for 20 min at room temperature in the dark.

Stained cell samples were analyzed using a Becton Dickinson ACCURI® C6 flow cytometer (Franklin Lakes, NJ, USA). Live cells stained by SYBR Green I were observed using the FL1 channel (excitation wavelength 485 nm, emission wavelength 535 nm). Dead cells stained by PI were observed using the FL3 channel (excitation wavelength 485 nm, emission wavelength 635 nm). Unstained cell samples were included as controls to exclude the signals from soil particles and debris. The threshold cutoff was set as 10,000, and each sample was run for 1 min in “slow” mode. PBS buffer was used as a blank control. Gating was used to separate positive signals from background noise. Flow cytometer settings were validated using E. coli as in Example 1. The gate of dead cells was usually interfered by the background noise, particularly in clay soils. Therefore, only live cells were reported.

Propidium Monoazide Treatment

Propidium monoazide (PMA) was used to remove DNA from cells without intact membranes. The PMA stock solution (10 mM) was prepared by dissolving 1 mg Biotium PMA (cat. NC9734120, Fisher Scientific) in 195.7 μl Dimethyl Sulfoxide (DMSO). To prepare soil slurries for PMA treatment, 0.5 g fresh soil was added to 50 ml PBS buffer and vortexed to mix the samples. Two ml of soil slurries or cell samples in triplicates were then transferred into transparent disposable cell culture tubes (Fisher Scientific, cat.14-956-3C). For PMA treatment group, 10 μl of PMA stock solution was added into the samples to a final PMA concentration of 50 μM Sterile water (10 μl) was added in control group samples. All samples were incubated for 5 min at room temperature in the dark and then exposed to LED light inside the hood of Azure Biosystems C400 (RGB-cy2, 470 nm) for 20 min with manual shaking every 5 min.

High-Throughput Amplicon Sequencing and Raw Data Processing

DNA was extracted from the original soil samples and soil extracted cells using QIAGEN PowerSoil DNA extraction kits (Qiagen, Germantown, MD) following the manufacturer's protocol and quantified using a NanoDrop spectrophotometer (NanoDrop Technologies, Wilmington, DE).

The V4 region of 16S rRNA gene was amplified using the primer pair and a two-step PCR as described in Example 1. The amplicons were sequenced (2×150 bp) on a MiSeq system (Illumina, San Diego, CA, USA). Sequences were processed using a Galaxy-based sequence analysis pipeline. The forward and reverse reads were first assigned to different samples based on the barcodes. The primer sequences were then trimmed and the Brim program was used to filter the reads with a threshold quality score greater than 20 within a 5 bp window size and a minimum length of 100 bp. Forward and reverse reads with at least a 50 bp overlap and less than 5% mismatches were j oined using FLASH. Sequences with ambiguous N bases were discarded. Joined sequences with lengths between 245 and 260 bp were clustered into operational taxonomic units (OTU) at the 97% identity using UPARSE. Singletons were removed. Then, taxonomic assignment was conducted through the RDP classifier with a confidence cutoff of 0.5. Sequences classified as Chloroplasts and Mitochondria were removed. An approximately-maximum-likelihood phylogenetic tree was constructed based on the representative sequences for each OTU using FastTree v.2.0.

Statistical Analyses

All statistical analyses were conducted in R software. The analysis of variance (ANOVA) and Tukey's honestly significant difference (HSD) were used to characterize the statistical significance of the differences between the soil cell extraction procedures. Microbial 16S sequencing data were organized for diversity analysis using the R package phyloseq. After raw data processing, the retained high-quality sequences were randomly resampled to a depth of 17320 reads per sample for 16S rRNA gene. Alpha diversity and beta diversity of the microbial communities were then calculated. Nonmetric multidimensional scaling (NMDS) and PERMANOVA were conducted to visualize and assess the Weighted UniFrac distance matrices, which incorporates both the relative abundance and phylogenetic information of each taxon, using the R package vegan.

Results

Comparison of Soil Cell Extraction Procedures

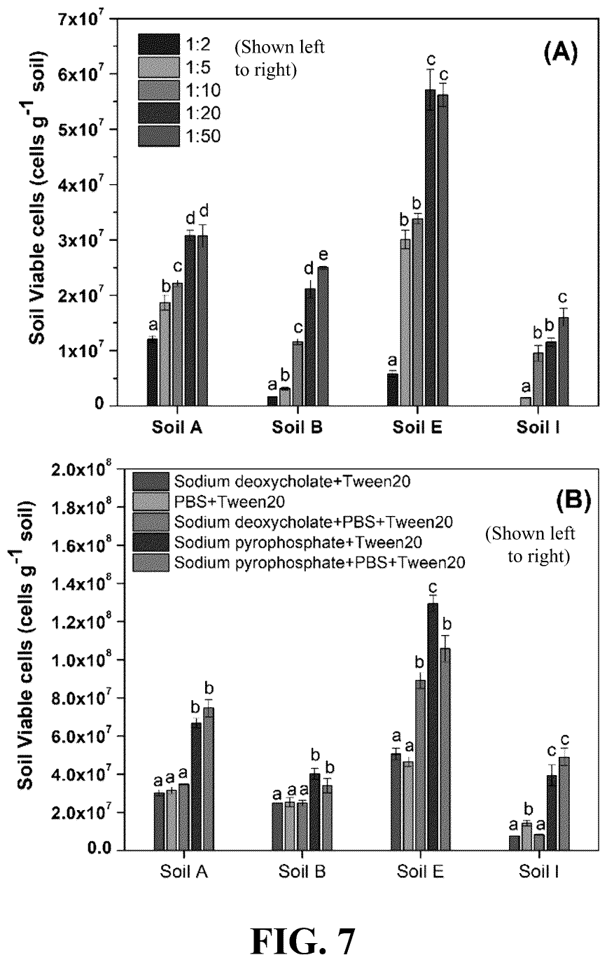

Five soil-to-buffer ratios including 1:2 (50 g soil :100 ml 0.5% TWEEN® 20 in PBS), 1:5, 1:10, 1:20, 1:50 were tested. The yield of soil extractable viable cells increased as the soil to buffer ratios increased. The soil to buffer ratios of 1:20 to 1:50 were suitable for a wide range of soil types. To get enough cells for downstream analysis, the soil to buffer ratio of 1:20 was used for soil cell extraction. Three blending times (3, 4, and 5 min) were also tested. The cell yield increased as blending time increased only for Soil A. Additionally, longer blending time resulted in lower viability of extracted cells. Results are shown in FIG. 7(a). Collectively, 3 min was concluded as the best blending time.

Five different chemical solutions were tested including: 0.1% sodium deoxycholate (SD), PBS buffer, 0.1% SD +PBS buffer, 50 mM sodium pyrophosphate, 50 mM sodium pyrophosphate+PBS buffer. Each solution was mixed with tween 20 to a final concentration of 0.5% tween 20. Compared with SD, PBS buffer, as well as the mixture of SD and PBS buffer, sodium pyrophosphate had significant higher soil viable cell yield. The addition of PBS to sodium pyrophosphate did not further increase viable cell yield. Results are shown in FIG. 7(b).

Recovery Evaluation of Soil Cell Extraction Procedures In Diverse Soils

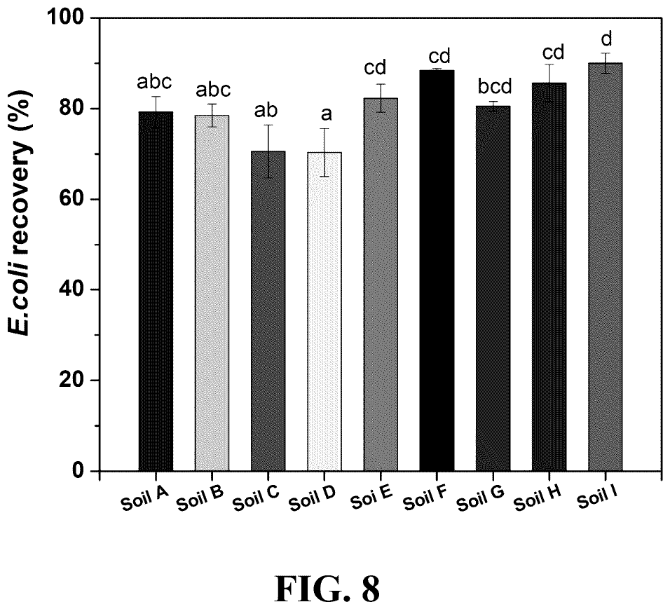

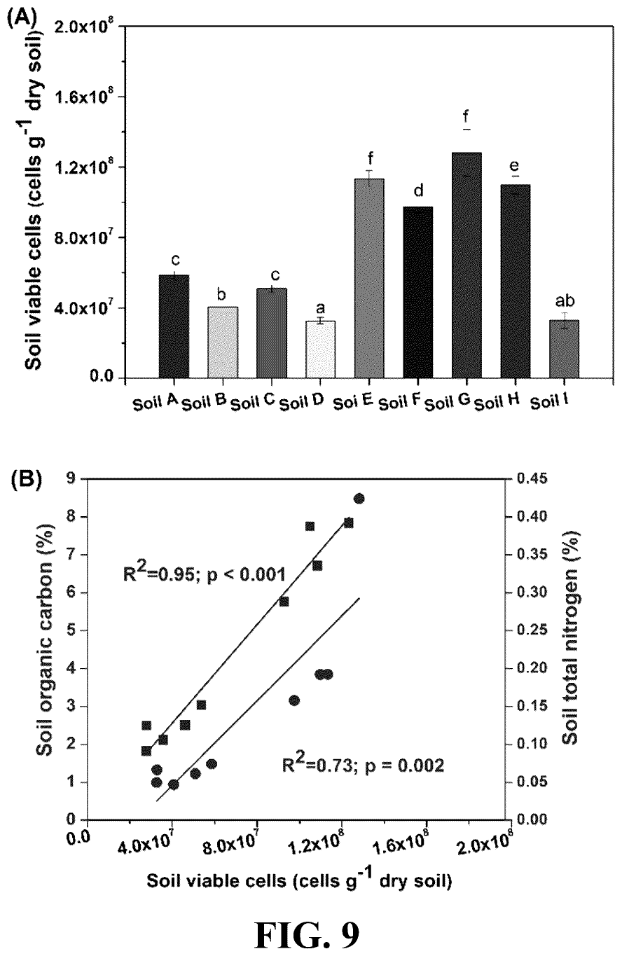

Using the disclosed cell extraction procedure, the recovery rates of spiked with E. coli in nine soils with different textures, including loam, sandy loam, silk loam, clay loam, and clay, were tested. The recovery rates of spiked in E coli were 70-90%, with the average of 80.6% across nine soils. The yield of soil viable cells ranged from 3.3×10⁷ to 1.3×10⁸ per gram of soil sample. Results are shown in FIG. 8. Forest soil (Soil G) and native prairie soil (Soil E) had higher soil viable cells than other soils. The yield of soil viable cells was significantly correlated with soil organic carbon or soil total nitrogen. Results are shown in FIG. 9.

Microbial Community Compositions In Original Soil, Extracted Cells, and Soil Pellet

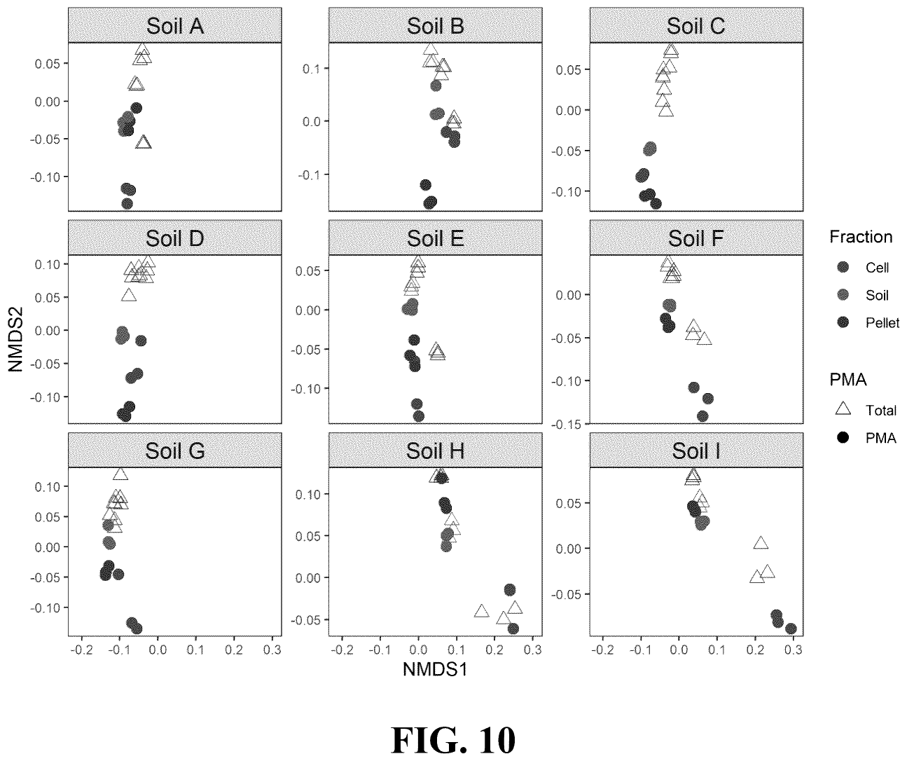

This Example also evaluated the microbial community compositions in original soil, extracted cells, and soil pellet after cell collection across nine soils. The microbial community compositions were different in these three fractions. Microbial communities of extracted cells were more dissimilar from the microbial community of original soils in silt loam and clay soils. PMA treatment (removal of DNA from cells without intact membranes) resulted in significantly different microbial communities from original soil samples (p<0.001), extracted cells (p<0.001), and soil pellet (p<0.001). The microbial richness of soil viable cells (with PMA treatment) was significantly different across nine soils (p<0.001). Soil E (native prairie soil) had highest richness, while soil A (grassland soil) and soil I (agricultural soil) had lowest richness. Compared with original soil samples, dominant phyla (>1%) of viable microbial communities (with PMA treatment) in soil cells were often not consistently changed across nine soils. Generally, lower relative abundance of Proteobacteria, Acidobacteria, Bacteroidetes, and Chloroflexi; but higher relative abundance of Crenarchaeota and Planctomycetes were observed in soil cells. Results are shown in FIG. 10.

Enzyme Treatment Increased the Yield and Diversity of Soil Viable Cells In the 2nd Round Extraction

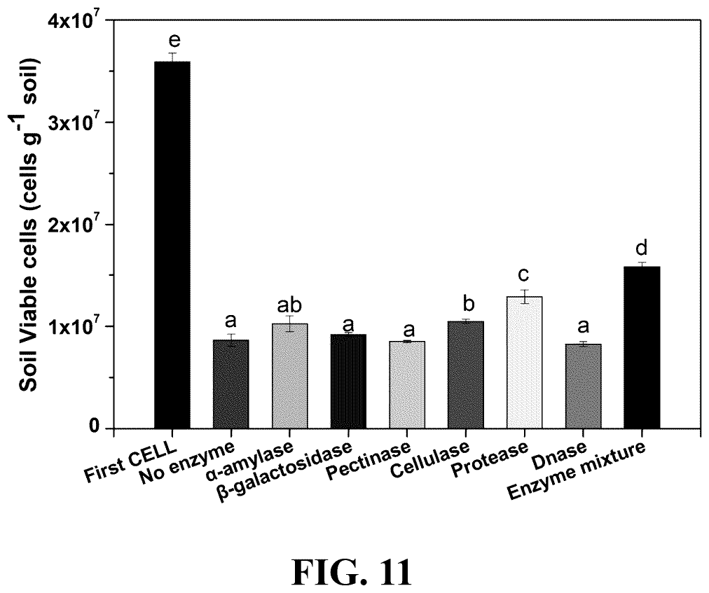

Before the 2^{nd} round of cell extraction, seven individual enzymes (α-amylase, β-galactosidase, pectinase, lipase, cellulase, protease, and DNase) or enzyme mixture (α-amylase, β-galactosidase, and cellulase) were added into the soil slurry and incubated for 30 min at 37° C. The lipase treatment had noise signal, which interfered with the gate of soil viable cells. Therefore, the soil yield in this treatment was not reported. Cellulase and protease treatment significantly increased the yield of soil extractable cells in the 2nd round extraction. Treatment with the enzyme mixture significantly increased the cell yield of soil extractable cells compared to individual enzyme. Results are shown in FIG. 11.

Soil Cell Extraction Procedure For Small-Scale Soil Samples

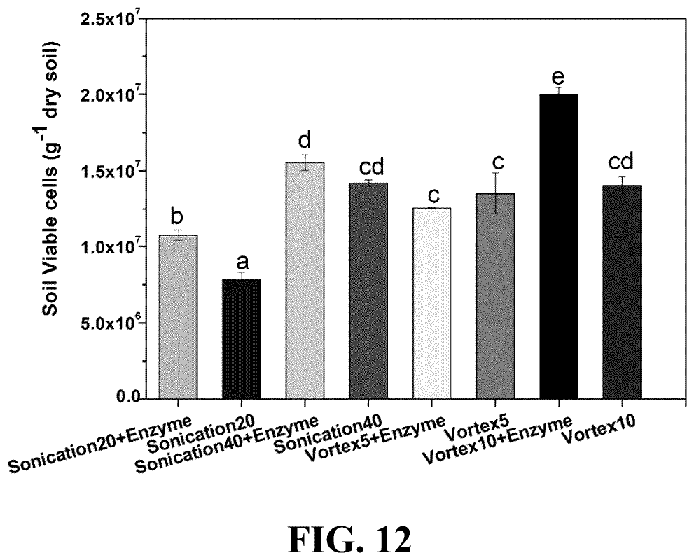

For small-scale soil samples, sonication and vortex were compared for physical dispersion, as blending cannot be applied due to the limitation of sample volume. 40 s sonication had a higher soil viable cell yield than 20 s sonication. There was no difference in soil viable cell yield between 5-min and 10-min vortex. Enzyme pre-treatments were also added before physical dispersion. It was found that pre-treatment with enzyme mixture significantly increased the soil viable cell yield in both sonication and vortex. Enzyme pretreatment and 10-min vortex had the highest cell yield among tested treatments. Results are shown in FIG. 12.

Discussion

Extraction of microbial cells from soil is a prerequisite step for the application of many microbial discovery campaigns, including those performed at single-cell resolution. Previous work on extraction methods often focused on the improvement of cell recovery efficiency evaluated at the total number of cells instead of total number of viable cells. In this study, it was examined how extraction procedures and soil storage conditions affected the viability and microbial community compositions of the soil extractable cells using four soil samples with different soil types (loam, sandy loam, and clay loam). The best combination of soil cell extraction methods of those tested, namely the use of blender+TWEEN® 20+80% NYCODENZ®, had the highest cell viability and yield among the conditions tested. Repeated cell extraction could improve the overall cell yield, but the viability could be compromised in the second round of cell extraction from soil. Enzyme pretreatment improved the yield of viable cells. These results show that there is a significant difference between total cells extracted and total viable cells extracted under all conditions tested in the present examples, and that for some microbiological applications, focusing on viable cell extraction is critical rather than total cells.

Soil cell extraction efficiency.

We synthesized the previous work on soil cell recovery rates. It was found that the indigenous bacteria had much lower extraction efficiency than spiked bacteria. The average of extraction efficiency of the indigenous bacteria was around 15% while the spiked bacteria was around 80%. Soil samples spiked with E. coli had a recovery rate of 71-90%, which was similar to Example 1. These results indicate that the key challenge of soil cell extraction was the dispersion step. Once the microbes were dispersed, they would be easily recovered by NYCODENZ® purification.

Soil properties

The yield of soil viable cells was significantly correlated with soil organic C and total N. Previous studies have shown that microbial biomass is associated with soil organic C and total N. It was also found that microbial communities of extracted cells were more dissimilar from the microbial community of original soils in silt loam and clay soils. For soil-to-solution ratios, it was found that clay soil and clay loam soil had highest yield of soil viable cells at the 1:50 ratio.

Enzymes

The present work has demonstrated that enzyme pretreatment improved the yield of soil viable cells. The enzyme treatment breaks down soil EPS, releasing EPS associated microorganisms. Cellulase and protease had better performance than other tested individual enzymes. Protein and cellulose were abundant organic compounds in soils. Protease may damage the cells and may impact the viability of microorganisms by disrupting the membrane, which is mainly constituted of proteins and lipids. A mixture of α-amylase, (β-galactosidase, and cellulase was developed, which had a better cell yield compared with other individual enzymes.

Conclusions

The yield of soil extractable viable cells increased as the soil to buffer ratios increased. A soil to buffer ratio of 1:20 was identified as the best ratio extract enough cells for downstream analysis. Sodium pyrophosphate with TWEEN° 20 had significantly higher soil viable cell yield. Cellulase and protease treatment significantly increased the yield of soil extractable cells in the 2nd round extraction. Treatment with an enzyme mixture significantly increased the cell yield of soil extractable cells compared to individual enzymes.

### Example 3

The following experiments were conducted to refine procedures.

Sample preparation: Five soil-to-buffer (0.5% Tween® 20 in PBS) ratios including 1:2 (50 g soil:100 ml buffer), 1:5 (20 g soil:100 ml buffer), 1:10 (10g soil :100 ml buffer), 1:20 (5g soil:100 ml buffer), 1:50 (2 g soil:100 ml buffer) were tested. Of the ratios tested, 1:20 provided the best soil to buffer ratio for the recovery of soil-extractable cells. The test was done with four types of soil samples.

Chemical dispersion: Five different chemical solutions were tested including: 0.1% sodium deoxycholate, PBS buffer, 0.1% SD+PBS buffer, 50 mM sodium pyrophosphate, 50 mM sodium pyrophosphate+PBS buffer. Each solution was mixed with TWEEN® 20 to a final concentration of 0.5% TWEEN® 20. Of the solutions tested, sodium pyrophosphate with 0.5% TWEEN® 20 resulted in the most cells recovered.

Enzyme treatment: Six individual enzymes (α-amylase, (β-galactosidase, pectinase, cellulase, protease, and DNase) or an enzyme mixture (α-Amylase, (β-Galactosidase, and Cellulase) were added into the soil slurry and incubated for 30 min at 37° C. It was found that enzyme pre-treatment increased microbial cell yield and pre-treatment with the enzyme mixture significantly increased the cell yield of soil extractable cells compared to pre-treatment with any single enzyme.

Physical dispersion: Blending recovered more total and viable extractable cells than vortexing and sonication. For small-scale soil samples, vortexing is a better choice than sonication for physical dispersion, as blending cannot be applied due to the limitation of sample volume.

Density gradient medium: Increasing the NYCODENZ® concentration 90% had little effect on the yield and viability of soil extractable cells. In at least certain embodiments the concentration of density gradient medium can be in a range of 75% to 76% to 77% to 78% to 79% to 80% to 81% to 82% to 83% to 84% to 85%.

Repeated extraction/purification procedures: Repeated extraction increased the yield but reduced the viability of the extracted cells.

### Example 4

In one non-limiting embodiment, the method of the present disclosure includes the following materials and steps.

Reagents

(1) PBS buffer (1L): To 800 ml ultrapure water, add the following chemicals: 8 g of NaCl, 200 mg of KCl, 1.44 g of Na₂HPO₄, 240 mg of KH₂PO₄, adjust solution to pH≈7.4, add ultrapure water to final volume 1 L. Autoclave. (2) Sodium pyrophosphate buffer with TWEEN® (0.5% v/v): Dissolve 12.3 g sodium pyrophosphate tetrabasic (P8010, Sigma-Aldrich) to 1L ultrapure water and autoclave; add 5 ml tween 20 (P9416, Sigma-Aldrich) to 1 L autoclaved sodium pyrophosphate buffer. Shake to mix. (3) NYCODENZ® (80% w/v): To 80 g NYCODENZ® (AXS-1002424, Cosmo Bio) add 100 ml ultrapure water, stir and heat to dissolve (which may require about 2 h). Autoclave.

Materials and Apparatus

Waring blender (22000 rpm) (cat. 14-509-7P in Fisher); 25 ml pipette; Oak Ridge centrifuge tubes; MACS® SmartStrainers (30 μm) (MACS Miltenyi Biotec); Avanti J-20 XP centrifuge.

Soil Dispersion:

Large-Scale Soil Samples

In the blender: (1) Add 5 g soil and 100 ml sodium pyrophosphate buffer with TWEEN® 20 (0.5% v/v) to the Waring blender. (2) Blender at 22,000 rpm speed for 3 min at 1 min interval with 1 min incubation on ice to cool the mixture. Rinse the blender with water once followed by one rinse with alcohol (70%) after each sample.

Small-Scale Soil Samples

Vortex: (1) Add 1 g soil, 20 ml sodium pyrophosphate buffer with TWEEN® 20 (0.5% v/v), and 1.2 ml enzyme mixture (α-Amylase, (β-Galactosidase, and Cellulase) to the 50 ml centrifuge tube. (2) Vortex at the maximum speed for 10 minutes.

NYCODENZ® density gradient centrifugation for soil:

Add 18 ml NYCODENZ® cushions (80% w/v, 1.42 g ml⁻¹) in 50 ml Oak Ridge centrifuge tubes, then load soil slurries (20 ml) slowly onto the top of NYCODENZ®; Weigh Oak Ridge centrifuge tubes with soil slurries in fine scale with difference between each pair of tubes less than <0.01g; Centrifuge at 15,000×g for 40 min at 4° C. with slow acceleration and deceleration; Carefully transfer supernatant above NYCODENZ® (i.e., the cell-containing band and the supernatant above the band) to a new sterile 50 ml centrifuge tube; Add 20 ml PBS buffer to the tube. Vortex to mix; Filter the mixture with PBS buffer using SmartStrainers (30 μm) into a new Oak Ridge centrifuge tubes to remove the debris; Centrifuge at 15,000×g for 15 min at 4° C. with slow acceleration and deceleration; Discard the supernatant. Resuspend the cell pellet in 5 ml of PBS buffer.

### Example 5

In one non-limiting embodiment, the method of the present disclosure includes the following materials and steps.

Reagents

PBS buffer (1L): To 800 ml ultrapure water, add the following chemicals: 8 g of NaCl, 200 mg of KCl, 1.44 g of Na₂HPO₄, 240 mg of KH₂PO₄, Adjust solution to pH≈7.4, Add ultrapure water to final volume 1 L. Autoclave.

Sodium pyrophosphate buffer with TWEEN® 20 (0.5% v/v): Dissolve 12.3 g sodium pyrophosphate tetrabasic (P8010, Sigma-Aldrich) to 1L ultrapure water and autoclave; Add 5 ml tween 20 (P9416, Sigma-Aldrich) to 1 L autoclaved sodium pyrophosphate buffer. Shake to mix.

NYCODENZ® (80% w/v): Add 80 g NYCODENZ® (AXS-1002424, Cosmo Bio) to 100 ml ultrapure water, stir and heat to dissolve (Caution: this may take about 2 h to dissolve). Autoclave.

Materials and Apparatus

(1) Waring blender (22000 rpm) (cat. 14-509-7P in Fisher), (2) 25 ml pipette, (3) Oak Ridge centrifuge tubes, (4) MACS® SmartStrainers (30 μm) (MACS Miltenyi Biotec), (5) Avanti J-20 XP centrifuge.

Soil Dispersion:

Large-Scale Soil Samples

In the blender: (1) Add 5 g soil and 100 ml sodium pyrophosphate buffer with TWEEN® 20 (0.5% v/v) to the Waring blender. (2) Blender at 22,000 rpm speed for 3 min at 1 min interval with 1 min incubation on ice to cool the mixture. Rinse the blender with water once followed by one rinse with alcohol (70%) after each sample.

Small-Scale Ssoil Samples

Vortex: (1) Add 1 g soil, 20 ml sodium pyrophosphate buffer with TWEEN® 20 (0.5% v/v), and 1.2 ml enzyme mixture (a-Amylase, (3-Galactosidase, and Cellulase) to the 50 ml centrifuge tube. (2) Vortex at the maximum speed for 10 minutes.

NYCODENZ® density gradient centrifugation for soil: (1) Add 18 ml NYCODENZ® cushions (80% w/v, 1.42 g ml⁻¹) in 50 ml Oak Ridge centrifuge tubes, then load soil slurries (20 ml) slowly onto the top of NYCODENZ®, (2) Weigh Oak Ridge centrifuge tubes with soil slurries in fine scale with difference between each pair of tubes less than <0.01 g, (3) Centrifuge at 15,000×g for 40 min at 4° C. with slow acceleration and deceleration, (4) Carefully transfer supernatant above NYCODENZ® to a new sterile 50 ml centrifuge tube, (5) Add 20 ml PBS buffer to the tube. Vortex to mix, (6) Filter the mixture with PBS buffer using SmartStrainers (30 μm) into a new Oak Ridge centrifuge tubes to remove the debris, (7) Centrifuge at 15,000×g for 15 min at 4° C. with slow acceleration and deceleration, and (8) Discard the supernatant. Resuspend the cell pellet in 5 ml of PBS buffer.

### Example 6

In one non-limiting embodiment, a large-scale soil sample comprising 4 g of fresh soil, and 80 ml of 50 mM sodium pyrophosphate mixed with 0.5% TWEEN® 20 were added to the Waring blender (ConairTM 7012S, cat. 14-509-7G) and blended at highest speed (22,000 rpm) for 3 min at 1 min interval with 1 min incubation on ice to cool the mixture. After dispersion, 20 ml of the soil slurry was slowly added on the top of 18 ml 80% NYCODENZ® in a 50 ml sterile Oak Ridge centrifuge tube and centrifuged at 15,000×g for 40 min at 4° C. with slow acceleration and deceleration (three replicates). The layer containing cells above the NYCODENZ® was then carefully collected using a 5 ml pipette and around 18 ml solution was transferred into a new sterile 50 ml tube. The PBS buffer was added to bring the total volume to 35 ml. The mixed solution was filtered with a sterile filter (MACS® SmartStrainers, 30 μm pore size, cat.130-098-458) into a new sterile Oak Ridge centrifuge tube containing 18 ml 80% NYCODENZ® to remove the large soil debris. Soil-extracted cells were pelleted by centrifuging at 15,000×g for 15 min at 4° C. with slow acceleration and deceleration. For short-term storage (1-3 days) at 4° C., 5 ml soil extracts could be added to resuspend soil-extracted cells.

### Example 7

In one non-limiting embodiment, a small-scale soil sample comprising 1 g soil and 20 ml sodium pyrophosphate mixed with 0.5% TWEEN® 20 were added to a 50 ml sterile Oak Ridge centrifuge tube. Then, 1 ml of a cocktail of enzymes (α-Amylase, (β-Galactosidase, and Cellulase) was added into the soil slurry and shaken for 60 minutes at 37° C. with a speed of 100 rpm. After enzyme treatment, the soil slurry was further dispersed by vortex with maximum speed for 10 min. The soil slurry was slowly added on the top of 18 ml 80% NYCODENZ® in a 50 ml sterile Oak Ridge centrifuge tube and centrifuged at 15,000×g for 40 min at 4° C. with slow acceleration and deceleration. The layer containing cells above the NYCODENZ® was then carefully collected using a 5 ml pipette and about 18 ml solution was transferred into a new sterile 50 ml tube. The PBS buffer was added to bring the total volume to 35 ml. The mixed solution was filtered with a sterile filter (MACS® SmartStrainers, 30 μm pore size, cat.130-098-458) into a new sterile Oak Ridge centrifuge tube containing 18 ml 80% NYCODENZ® to remove the large soil debris. Soil-extracted cells were pelleted by centrifuging at 15,000×g for 15 min at 4° C. with slow acceleration and deceleration. For short-term storage (1-3 days) at 4° C., 5 ml soil extracts could be added to resuspend soil-extracted cells.

### Example 8

An extraction kit comprising 4-5 solutions, 50 ml centrifuge tubes, and 30 um filters was developed. The soil cell extraction kit was applied to nine soils with different textures, including loam, sandy loam, silk loam, clay loam, and clay. The cell recovery rates of soil samples spiked with E. coli were 70-90%, with the average of 80.6% across nine soils. The yield of viable cells ranged from 5×10⁷ to 4×10⁸ per gram of soil sample. The yield of viable cells was significantly correlated with soil organic carbon or soil total nitrogen. Collectively, the results indicated that the soil cell extraction kit can be applied for isolation of viable cells from diverse soils.

In one non-limiting example, the kit contains the following components:

1. One or more containers comprising sodium pyrophosphate (e.g., 50 mM) with a surfactant (e.g., 0.5% Tween® 20),

2. One or more containers comprising an enzyme mixture of α-Amylase (e.g., 500 units), (β-Galactosidase (e.g., 250 units), and Cellulase (e.g., 600 units),

3. One or more containers comprising a density gradient medium (e.g., 80% (w/v) NYCODENZ)®,

4. One or more containers comprising PBS buffer, and

5. One or more sterile filters (e.g., MACS® SmartStrainers, 30 μm pore size).

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain non-limiting embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus, the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components and compositions described herein, the methods described herein, or in the steps or the sequence of steps of the methods described herein, without departing from the spirit and scope of the present disclosure.

What is claimed is:

1. A method of extracting viable bacterial cells from a soil sample, the method comprising the steps of:
mixing a dispersant-surfactant solution with a soil sample to form a soil slurry;
adding the soil slurry to a centrifuge tube containing a density gradient medium;
centrifuging the centrifuge tube to form a solvent layer above the density gradient medium, wherein the solvent layer comprises viable bacterial cells;
extracting the solvent layer to form an extracted solvent layer containing the viable bacterial cells;
combining the extracted solvent layer with a phosphate buffered saline (PBS) solution to form a PBS-cell mixture;
filtering the PBS-cell mixture to form a cell filtrate;
depositing the cell filtrate into a second centrifuge tube containing a quantity of the density gradient medium;
centrifuging the second centrifuge tube to form a second solvent layer above the density gradient medium contained in the second centrifuge tube; and
extracting the second solvent layer from the second centrifuge medium to form a second cell filtrate comprising the viable bacterial cells.

2. The method of claim 1, wherein the soil sample is at least 2.5 g.

3. The method of claim 1, wherein the soil sample is less than 2.5 g.

4. The method of claim 1, wherein the density gradient medium is a non-ionic density gradient medium.

5. The method of claim 4, wherein the non-ionic density gradient medium comprises 5-(N-2,3 -dihydroxypropylacetamido)-2,4,6-tri-iodo-N,N′-bis (2,3 dihydroxypropyl) isophthalamide.

6. The method of claim 1, wherein the dispersant of the dispersant-surfactant solution is selected from sodium pyrophosphate, sodium deoxycholate, sodium phosphate, potassium citrate buffer, phosphate buffered saline (PBS), and glycerol.

7. The method of claim 1, wherein the surfactant of the dispersant-surfactant solution is an ionic surfactant.

8. The method of claim 1, wherein the surfactant of the dispersant-surfactant solution is a non-ionic surfactant.

9. A method of extracting viable bacterial cells from a soil sample, the method comprising the steps of:
mixing a dispersant-surfactant solution with a soil sample to form a soil slurry;
combining the soil slurry with an enzyme mixture with forming a slurry-enzyme mixture;
shaking the slurry-enzyme mixture;
vortexing the shaken slurry-enzyme mixture forming a vortexed slurry-enzyme mixture;
adding the vortexed slurry-enzyme mixture to a centrifuge tube comprising a density gradient medium;
centrifuging the centrifuge tube to form a solvent layer above the density gradient medium, wherein the solvent layer comprising viable bacterial cells;
extracting the solvent layer from the centrifuge tube to form an extracted solvent layer comprising the viable bacterial cells;
combining the extracted solvent layer with a phosphate buffered saline (PBS) solution to form a PBS-cell mixture;
filtering the PBS-cell mixture to form a cell filtrate;
depositing the cell filtrate into a second centrifuge tube comprising the density gradient medium;
centrifuging the second centrifuge tube forming a second solvent layer above the density gradient medium; and
extracting the solvent layer from the second centrifuge medium forming a second cell filtrate comprising the viable bacterial cells.

10. The method of claim 9, wherein the enzyme mixture comprises α-Amylase, (β-Galactosidase, and Cellulase.

11. The method of claim 9, wherein the enzyme mixture consists of α-Amylase, (β-Galactosidase, and Cellulase.

12. The method of claim 9, wherein the soil sample is at least than 2.5 g.

13. The method of claim 9, wherein the soil sample is less than 2.5 g.

14. The method of claim 9, wherein the density gradient medium is a non-ionic density gradient medium.

15. The method of claim 14, wherein the non-ionic density gradient medium comprises 5-(N-2,3-dihydroxypropylacetamido)-2,4,6-tri-iodo-N,N′-bis (2,3 dihydroxypropyl) isophthalamide.

16. The method of claim 9, wherein the dispersant of the dispersant-surfactant solution is selected from sodium deoxycholate, sodium phosphate, sodium pyrophosphate, potassium citrate buffer, phosphate buffered saline (PBS), and glycerol.

17. The method of claim 9, wherein the surfactant of the dispersant-surfactant solution is an ionic surfactant.

18. The method of claim 9, wherein the surfactant of the dispersant-surfactant solution is a non-ionic surfactant.


SEQUENCE LISTING (2 sequences)

SEQ ID NO: 1 - DNA, 19 bp, synthetic construct
  source 1..19: mol_type = other DNA
gtgccagcmgccgcggtaa

SEQ ID NO: 2 - DNA, 20 bp, synthetic construct
  source 1..20: mol_type = other DNA
ggactachvgggtwtctaat